(19) United States
(12) Patent Application Publication
(10) Pub. No.: US 2025/0057862 A1
(43) Pub. Date: Feb. 20, 2025

(54) SIALIC ACID COMPOSITIONS FOR USE IN INHIBITING AND TREATING INFECTIONS IN FISH

(71) Applicant: LifeScience AS, Orsta (NO)

(72) Inventors: Jan Remmereit, Hovdebygda (NO); Trond-inge Kvernevik, Orsta (NO)

(21) Appl. No.: 18/720,169

(22) PCT Filed: Dec. 15, 2022
(86) PCT No.: PCT/IB2022/000781

§ 371 Date: Jun. 14, 2024

Related U.S. Application Data
(60) Provisional application No. 63/290,953, filed on Dec. 17, 2021.

(51) Int. Cl. A61K 31/7012; A23K 10/22; A23K 10/30; A23K 20/158; A23K 20/163; A23K 50/80; A61P 31/14; A61P 33/14

(52) U.S. Cl. CPC A61K 31/7012; A23K 10/22; A23K 10/30; A23K 20/158; A23K 20/163; A23K 50/80; A61P 31/14; A61P 33/14

(57) ABSTRACT

The present invention relates to the use of compositions comprising sialic acid to improve the health of fish in aquaculture, and in particular to inhibit or treat viral infections in fish, improve growth, and to inhibit or treat infestation of fish with sea lice.

### CROSS REFERENCE TO RELATED APPLICATIONS

This application claims the benefit of U.S. Prov. Appl. 63/290,953 filed Dec. 17, 2021, the entire contents of which are incorporated herein by reference.

### FIELD OF THE INVENTION

The present invention relates to the use of compositions comprising sialic acid to improve the health of fish in aquaculture, and in particular to inhibit or treat viral infections in fish, improve growth, and to inhibit or treat infestation of fish with sea lice.

### BACKGROUND OF THE INVENTION

Viral diseases and sea lice are major, complex and unresolved challenges for the salmon industry, and there is a strong need for solutions that improve fish welfare and prevent fish death and premature slaughter.

Salmon pancreas disease virus (SPDV), often referred to as salmonid alphavirus (SAV), causes pancreas disease (PD) in European salmonids. It is a highly contagious virus and the etiological agent of pancreas disease (PD) in marine reared Atlantic salmon (Salmo salar) and rainbow trout (Oncorhynchus mykiss), and referred to as sleeping disease (SD) in freshwater reared rainbow trout. SAV transmits horizontally from fish shedding virus into the water and ocean currents are believed to be a main contributor of viral spread between marine farms.

Infectious salmon anemia virus (ISAV, also referred to as Salmon isavirus) causes severe anemia in infected fish. Unlike the mature red blood cells of mammals, the mature red blood cells of fish contain DNA, and can become infected by viruses. The fish develop pale gills, and may swim close to the water surface, gulping for air. However, the fish may show no external signs of illness and maintain a normal appetite, until suddenly dying. The disease can progress slowly throughout an infected farm and, in the worst cases, death rates may approach 100%. Post-mortem examination of the fish has shown a wide range of causes of death. The liver and spleen may be swollen, congested or partially already dead. The circulatory system may stop working, and the blood may be contaminated with dead blood cells. Red blood cells still present burst easily, and the numbers of immature and damaged blood cells are increased. Infectious salmon anemia appears to be most like influenza viruses. Its mode of transfer and the natural reservoirs of infectious salmon anemia virus are not fully understood. Apart from Atlantic salmon, both sea-run Brown trout (Salmo trutta) and Rainbow trout (Onchorhyncus mykiss) can be infected, but do not become sick, so it is thought possible that these species may act as notable carriers and reservoirs of the virus.

IPN (Infectious Pancreatic Necrosis) is a contagious viral infection in salmonids caused by Infectious Pancreatic Necrosis Virus (IPNV) that has been prevalent in Norway. Outbreaks lead to a weakening of the digestive system and affected fish can be undernourished and weakened, and can die in the worst case scenario. The Norwegian Veterinary institute also notes that fish that have undergone IPNV infection are more susceptible to SPDV and HSMI.

HSMI (Heart and Skeletal Muscle Inflammation) is a disease that is connected with infection with Piscine Orthoreovirus (PRV). HSMI often affects salmon in the course of their first year at sea and mortality varies up to 20%. The highest proportion of mortality is often seen in connection with operational procedures, as these expose the circulatory system of the fish to extra stress. In addition to the increased mortality, an outbreak of HSMI may lead to reduced growth. Fish affected by disease often have a poor appetite, and in addition, some of the energy they receive will go towards fighting the disease. This can therefore lead to poorer utilization of the feed, an extended production period and therefore increased production costs.

CMS (Cardiomyopathy Syndrome) is caused by Piscine Myocarditis Virus (PMCV). The disease is characterized by the infiltration of large numbers of inflammatory cells into the heart muscles. CMS outbreaks can lead to substantial financial losses for fish farmers as they affect salmon as they approach their harvest size. In particular, PMCV-infected fish are susceptible to mortality when subjected to stressful conditions such as frequent sorting, mechanical de-lousing and transportation in live carriers.

Sea lice is a parasite that can infect all salmon fish by living naturally in salt water. In areas with high levels of aquaculture, there has been an increase in the number of sea lice in the water in recent years. The lice eat salmon skin, mucus and blood, and the fish can thus develop large wounds that can lead to reduced growth and a weakened health condition. In addition, infections can occur in the wounds. In the worst case, the fish die. Salmon are most vulnerable to lice when they are small. Salmon lice are a major indirect cause of death in adult farmed salmon. Chemical or mechanical de-lousing precipitate death in large numbers of fish with underlying viral infections such as PMCV, and weakening of the mucosal barrier as well as cuts and bruises from the handling renders the fish vulnerable to both bacterial and viral infections.

Further, various issues with growth of fish in aquaculture have been noted. These issues include declining feed conversion ratio (FCR) as well as issues with arrested growth after transfer of smolt to sea cages.

What is needed in the art are feed supplements that can reduce or treat infection in aquacultured fish, as well as improved various parameters associated with growth.

### SUMMARY OF THE INVENTION

The present invention relates to the use of compositions comprising sialic acid to improve the health of fish in aquaculture, and in particular to inhibit or treat viral infections in fish, improve growth, and to inhibit or treat infestation of fish with sea lice.

In some preferred embodiments, the present invention provides methods for treating or inhibiting infection by Infectious Salmon Anemia Virus (ISAV), in fish in aquaculture, the method comprising: administering a composition comprising sialic acid or precursor thereof in an effective concentration to the fish under conditions such that infection by ISAV is treated.

In some preferred embodiments, the present invention provides methods for prophylaxis of infection by ISAV, in fish in aquaculture, the method comprising: administering a composition comprising sialic acid or precursor thereof in an effective concentration to the fish under conditions such that infection by ISAV is inhibited.

In some preferred embodiments, the present invention provides methods for treating or inhibiting infection by Salmon Pancreas Disease Virus (SPDV), in fish in aquaculture, the method comprising: administering a composition comprising sialic acid or precursor thereof in an effective concentration to the fish under conditions such that infection by SPDV is treated.

In some preferred embodiments, the present invention provides methods for prophylaxis of infection by SPDV, in fish in aquaculture, the method comprising: administering a composition comprising sialic acid or precursor thereof in an effective concentration to the fish under conditions such that infection by SPDV is inhibited.

In some preferred embodiments, the present invention provides methods for treating or inhibiting infection by sea lice, in fish in aquaculture, the method comprising: administering a composition comprising sialic acid or precursor thereof in an effective concentration to the fish under conditions such that infection by sea lice is treated.

In some preferred embodiments, the present invention provides methods for prophylaxis of infection by sea lice, in fish in aquaculture, the method comprising: administering a composition comprising sialic acid or precursor thereof in an effective concentration to the fish under conditions such that infection by sea lice is inhibited.

In some preferred embodiments, the present invention provides methods for treating or inhibiting infection by Infectious Pancreatic Necrosis Virus (IPNV), in fish in aquaculture, the method comprising: administering a composition comprising sialic acid or precursor thereof in an effective concentration to the fish under conditions such that infection by IPNV is treated.

In some preferred embodiments, the present invention provides methods for prophylaxis of infection by IPNV, in fish in aquaculture, the method comprising: administering a composition comprising sialic acid or precursor thereof in an effective concentration to the fish under conditions such that infection by IPNV is inhibited.

In some preferred embodiments, the present invention provides methods for treating or inhibiting infection by Piscine Orthoreovirus (PRV), in fish in aquaculture, the method comprising: administering a composition comprising sialic acid or precursor thereof in an effective concentration to the fish under conditions such that infection by PRV and/or heart and skeletal muscle inflammation is treated.

In some preferred embodiments, the present invention provides methods for prophylaxis of infection by PRV, in fish in aquaculture, the method comprising: administering a composition comprising sialic acid or precursor thereof in an effective concentration to the fish under conditions such that infection by PRV and/or heart and skeletal muscle inflammation is inhibited.

In some preferred embodiments, the present invention provides methods for treating or inhibiting infection by Piscine Myocarditis Virus (PMV or PMCV), in fish in aquaculture, the method comprising: administering a composition comprising sialic acid or precursor thereof in an effective concentration to the fish under conditions such that infection by PMV and/or cardiomyopathy syndrome is treated.

In some preferred embodiments, the present invention provides methods for prophylaxis of infection by PMV, in fish in aquaculture, the method comprising: administering a composition comprising sialic acid or precursor thereof in an effective concentration to the fish under conditions such that infection by PMV and/or cardiomyopathy syndrome is inhibited.

In some preferred embodiments, the present invention provides a sialic acid or precursor thereof for use in treating infection by ISAV in a fish in aquaculture.

In some preferred embodiments, the present invention provides a sialic acid or precursor thereof for use in prophylaxis of ISAV infection in a fish in aquaculture.

In some preferred embodiments, the present invention provides a sialic acid or precursor thereof for use in treating infection by SPDV in a fish in aquaculture.

In some preferred embodiments, the present invention provides a sialic acid or precursor thereof for use in prophylaxis of SPDV infection in a fish in aquaculture.

In some preferred embodiments, the present invention provides a sialic acid or precursor thereof for use in treating infection by sea lice in a fish in aquaculture.

In some preferred embodiments, the present invention provides a sialic acid or precursor thereof for use in prophylaxis of sea lice infection in a fish in aquaculture.

In some preferred embodiments, the fish is infected with ISAV. In some preferred embodiments, the fish is infected with SPDV. In some preferred embodiments, the fish is infected with sea lice. In some preferred embodiments, the fish is infected with PMV. In some preferred embodiments, the fish is infected with PRV. In some preferred embodiments, the fish is infected with IPNV.

In some preferred embodiments, the sialic acid or precursor thereof is elected from the group consisting of neuraminic acid, n-acetylneuraminic acid (NANA), n-glycolylneuraminic acid (NGNA), and N-Acetyl-D-mannosamine. In some preferred embodiments, the sialic acid is NANA. In some preferred embodiments, the sialic acid is NGNA. In some preferred embodiments, the sialic acid is neuraminic acid. In some preferred embodiments, the sialic acid precursor is N-Acetyl-D-mannosamine. In some preferred embodiments, the sialic acid or precursor thereof is a combination of two or more of NANA, NGNA, neuraminic acid and N-Acetyl-D-mannosamine in any combination. In some preferred embodiments, the sialic acid or precursor thereof is a purified sialic acid or precursor thereof.

In some preferred embodiments, the sialic acid or precursor thereof is administered orally. In some preferred embodiments, the sialic acid or precursor thereof is administered topically. In some preferred embodiments, the sialic acid or precursor thereof is administered via a bath.

In some preferred embodiments, the sialic acid or precursor thereof is administered in a salmon feed. In some preferred embodiments, the salmon feed comprises from 0.05% to 2.0% w/w sialic acid or precursor thereof. In some preferred embodiments, the salmon feed comprises from 0.1% to 2.0% w/w sialic acid or precursor thereof. In some preferred embodiments, the salmon feed comprises from 0.5% to 1.5% w/w sialic acid or precursor thereof. In some preferred embodiments, the salmon feed further comprises a protein component, a carbohydrate component and a lipid component. In some preferred embodiments, the protein component is fish meal and/or krill meal. In some preferred embodiments, the carbohydrate component is a plant material. In some preferred embodiments, the lipid component is a fish oil or plant oil.

In some preferred embodiments, the present invention provides sialic acid or precursor thereof for use in effecting a health parameter in an aquatic animal, the health parameter selected from the group consisting of
a) prophylaxis of infection by a sialic acid-binding virus;
b) treatment of infection by a sialic acid-binding virus;
c) amelioration of symptoms caused by sea lice infestation;
d) improvement of skin barrier function by increasing sialic acid content in skin mucous;
e) improvement of gut barrier function by increasing sialic acid content in gut mucous;
f) increasing KDN content in the gut mucous;
g) decreasing stress levels as evidenced by a decrease in lactate levels;
h) treatment or prophylaxis of infection by Aeromonas salmonicida;
i) treatment of symptoms of furunculosis;
j) treatment or prophylaxis of infection by amoebas;
k) stimulation of mucous epithelia cells;
l) improvement in condition factor after smoltifying;
m) improvement of growth after smoltifying; and
n) improvement in length after smoltifying.

In some preferred embodiments, the present invention provides a method of improving a health parameter in an aquatic animal comprising administering to the aquatic animal sialic acid or a precursor thereof, the health parameter selected from the group consisting of
a) prophylaxis of infection by a sialic acid-binding pathogen;
b) treatment of infection by a sialic acid-binding pathogen;
c) amelioration of symptoms caused by sea lice infestation;
d) improvement of skin barrier function by increasing sialic acid content in skin mucous;
e) improvement of gut barrier function by increasing sialic acid content in gut mucous;
f) increasing KDN content in the gut mucous;
g) decreasing stress levels as evidenced by a decrease in lactate levels;
h) stimulation of mucous epithelia cells;
i) improvement in condition factor after smoltifying;
j) improvement of growth after smoltifying; and
k) improvement in length after smoltifying.

In some preferred embodiments, the health parameter is prophylaxis of infection by a sialic acid-binding pathogen. In some preferred embodiments, the health parameter is treatment of infection by a sialic acid-binding pathogen. In some preferred embodiments, the health parameter is amelioration of symptoms caused by sea lice infestation. In some preferred embodiments, the health parameter is improvement of skin barrier function by increasing sialic acid content in skin mucous. In some preferred embodiments, the health parameter is improvement of gut barrier function by increasing sialic acid content in gut mucous. In some preferred embodiments, the health parameter is increasing KDN content in the gut mucous. In some preferred embodiments, the health parameter is decreasing stress levels as evidenced by a decrease in lactate levels. In some preferred embodiments, the health parameter is stimulation of mucous epithelia cells. In some preferred embodiments, the health parameter is improvement in condition factor after smoltifying. In some preferred embodiments, the health parameter is improvement of growth after smoltifying. In some preferred embodiments, the health parameter is improvement in length after smoltifying.

In some preferred embodiments, two or more of health parameters a) to k) are improved. In some preferred embodiments, three or more of health parameters a) to k) are improved. In some preferred embodiments, four or more of health parameters a) to k) are improved. In some preferred embodiments, five or more of health parameters a) to k) are improved. In some preferred embodiments, six or more of health parameters a) to k) are improved. In some preferred embodiments, seven or more of health parameters a) to k) are improved. In some preferred embodiments, eight or more of health parameters a) to k) are improved. In some preferred embodiments, nine or more of health parameters a) to k) are improved. In some preferred embodiments, ten or more of health parameters a) to k) are improved.

In some preferred embodiments, the sialic acid or precursor thereof is selected from the group consisting of neuraminic acid, n-acetylneuraminic acid (NANA), n-glycolylneuraminic acid (NGNA), N-Acetyl-D-mannosamine, N-acetylglucosamine, mannosamine, fructose and mannose. In some preferred embodiments, the sialic acid is NANA. In some preferred embodiments, the sialic acid is NGNA. In some preferred embodiments, the sialic acid is neuraminic acid. In some preferred embodiments, the sialic acid precursor is N-Acetyl-D-mannosamine. In some preferred embodiments, the sialic acid precursor is N-acetylglucosamine. In some preferred embodiments, the sialic acid precursor is mannosamine. In some preferred embodiments, the sialic acid precursor is mannose. In some preferred embodiments, the sialic acid precursor is fructose.

In some preferred embodiments, the sialic acid or precursor thereof is a combination of two or more of neuraminic acid, n-acetylneuraminic acid (NANA), n-glycolylneuraminic acid (NGNA), N-Acetyl-D-mannosamine, N-acetylglucosamine, mannosamine, fructose and mannose in any combination.

In some preferred embodiments, the sialic acid or precursor thereof is a purified sialic acid or precursor thereof.

In some preferred embodiments, the sialic acid or precursor thereof is a free sialic acid.

In some preferred embodiments, the aquatic animal is a fish. In some preferred embodiments, the fish is a salmon. In some preferred embodiments, the aquatic animal is a shrimp.

In some preferred embodiments, the sialic acid is provided to smolts or after smoltification. Smoltification is the process where salmon parr undergo behavioral, developmental, and physiological changes into smolt, which in wild fish enable their first migration downstream to the sea and in farmed fish to be transferred primarily into floating net cages in the sea.

In some preferred embodiments, the sialic acid or precursor thereof is administered orally.

In some preferred embodiments, the sialic acid or precursor thereof is administered in a fish feed. In some preferred embodiments, the fish feed comprises from 0.01% to 2.0% w/w sialic acid or precursor thereof. In some preferred embodiments, the fish feed comprises from 0.02% to 2.0% w/w sialic acid or precursor thereof. In some preferred embodiments, the fish feed comprises from 0.05% to 1.5% w/w sialic acid or precursor thereof. In some preferred embodiments, the fish feed comprises from 0.01% to 0.5% w/w sialic acid or precursor thereof. In some preferred embodiments, the fish feed comprises from 0.05% to 0.5% w/w sialic acid or precursor thereof. In some preferred embodiments, the fish feed comprises from 0.02% to 0.5% w/w sialic acid or precursor thereof. In some preferred embodiments, the fish feed comprises from 0.07% to 0.25% w/w sialic acid or precursor thereof. In some preferred embodiments, the fish feed is supplemented with 0.01% to 2.0% w/w of a free sialic acid (i.e., non-conjugated sialic acid) or precursor thereof. In some preferred embodiments, the fish feed is supplemented with from 0.02% to 2.0% w/w free sialic acid or precursor thereof. In some preferred embodiments, the fish feed is supplemented with from 0.05% to 1.5% w/w of a free sialic acid or precursor thereof. In some preferred embodiments, the fish feed is supplemented with from 0.01% to 0.5% w/w of a free sialic acid or precursor thereof. In some preferred embodiments, the fish feed is supplemented with from 0.05% to 0.5% w/w of a free sialic acid or precursor thereof. In some preferred embodiments, the fish feed is supplemented with from 0.02% to 0.5% w/w of a free sialic acid or precursor thereof. In some preferred embodiments, the fish feed is supplemented with from 0.07% to 0.25% w/w of a free sialic acid or precursor thereof.

In some preferred embodiments, the fish feed further comprises a protein component, a carbohydrate component and a lipid component. In some preferred embodiments, the protein component is fish meal and/or krill meal. In some preferred embodiments, the carbohydrate component is a plant material. In some preferred embodiments, the lipid component is a fish oil.

In some preferred embodiments, the sialic acid or precursor thereof is administered topically. In some preferred embodiments, the sialic acid or precursor thereof is administered via a bath.

In some preferred embodiments, the sialic-acid binding pathogen is selected from the group consisting of a virus, a bacteria and an amoeba.

In some preferred embodiments, the sialic-acid binding pathogen is a bacteria. In some preferred embodiments, the bacteria is Aeromonas salmonica. In some preferred embodiments, the administration of the sialic acid or precursor thereof ameliorates one or more symptoms of furunculosis.

In some preferred embodiments, the sialic-acid binding pathogen is an amoeba. In some preferred embodiments, the amoeba is a Neoparamoeba. In some preferred embodiments, the administration of the sialic acid or precursor thereof ameliorates one or more symptoms of amoebic gill disease.

In some preferred embodiments, the sialic-acid binding pathogen is a virus. In some preferred embodiments, the virus is a non-enveloped icosahedral virus. In some preferred embodiments, the virus is ISAV. In some preferred embodiments, the administration of sialic acid or a precursor thereof ameliorates one or more symptoms associated with ISAV infection.

In some preferred embodiments, the virus is SPDV. In some preferred embodiments, the administration of sialic acid or a precursor thereof ameliorates one or more symptoms associated with SPDV infection.

In some preferred embodiments, the virus is IPNV. In some preferred embodiments, the administration of sialic acid or a precursor thereof ameliorates one or more symptoms associated with pancreatic disease.

In some preferred embodiments, the virus is PRV. In some preferred embodiments, the administration of sialic acid or a precursor thereof ameliorates one or more symptoms associated with heart and skeletal inflammation disease.

In some preferred embodiments, the virus is PMV. In some preferred embodiments, the administration of sialic acid or a precursor thereof ameliorates one or more symptoms associated with cardiomyopathy disease.

In some preferred embodiments, the virus is Alloherpesviridae.

In some preferred embodiments, the aquatic animal is infected with the virus.

### Definitions

As used herein, the term “inhibits” when used in reference to infection by a given virus refers to a reduction in infection in subjects such as fish exposed to the given virus.

“Administering” or “administration of” a substance, a compound or an agent to a subject can be carried out using one of a variety of methods known to those skilled in the art. For example, sialic acid can be administered, intravenously, arterially, intradermally, intra-muscularly, intraperitoneally, intravenously, subcutaneously, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

A “therapeutically effective amount” or a “therapeutically effective dose” of a drug or agent, such as sialic acid, is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of symptoms of the condition being treated. The skilled worker can readily determine the effective amount for a given situation by routine experimentation.

A “prophylactically effective amount” or a “prophylactically effective dose” of a drug or agent, such as sialic acid, is an amount of a drug or an agent that, when administered to a subject will have the intended prophylactic effect. The full prophylactic effect does not necessarily occur by administration of one dose, and may occur only after administration of a series of doses. Thus, a prophylactically effective amount may be administered in one or more administrations. The precise effective amount needed for a subject will depend upon, for example, the subject's size, health and age, the nature and extent of symptoms of the condition being treated. The skilled worker can readily determine the effective amount for a given situation by routine experimentation.

“Treating” a condition or patient refers to taking steps to obtain beneficial or desired results, including clinical results. Beneficial or desired clinical results include, but are not limited to, alleviation, amelioration, or slowing the progression, of one or more symptoms associated with viral infection. In certain embodiments, treatment may be prophylactic.

### BRIEF DESCRIPTION OF THE DRAWINGS

FIG. 1. Graph of data comparing ISAV detection in heart of treated and control fish.

FIG. 2. Graph of data comparing ISAV detection in kidney of treated and control fish.

FIG. 3. Graph of data comparing ISAV detection in treated and control fish.

FIG. 4. Schematic depiction of experimental design related to Example 3.

FIG. 5. Free and total Neu5Ac in the mucus layer on the skin of postsmolts in the saline phase (Day 78).

FIG. 6. Free and total Neu5Ac in the mucus layer in the gut of postsmolts in the saline phase (Day 78).

FIG. 7. Free and total Neu5Glc in mucus on skin (upper row) and intestine (lower row).

FIG. 8. Free and total Neu5Glc (upper row) and KDN (lower row) in the mucus layer in the intestine of fresh postsmolt in the seawater phase (Day 78)

FIG. 9. Free and total SA in plasma of postsmolt in saltwater phase (Day 78).

FIG. 10. Free and total Neu5Ac in the mucus layer on the skin of healthy fish on the control and SA diet.

FIG. 11. Free and total Neu5Ac in the mucus layer in the intestine of healthy fish on control and SA diets.

FIG. 12. Free and total Neu5Glc in the mucus layer on the skin of healthy fish on the control and SA diet.

FIG. 13. Free and total Neu5Glc in the intestinal mucus layer of healthy fish on control and SA diets.

FIG. 14. Free and total KDN in the mucus layer on the skin of healthy fish on the control and SA diet.

FIG. 15. Free and total KDN in the intestinal mucus layer of healthy fish on control and SA diets.

FIG. 16. Qualitative overview of changes in skin and gill epithelium from small presmolt to postsmolt.

FIG. 17. Average fitness factor, size and density of mucus cells in skin with different diets; fresh smolt/post smolt in sea phase.

FIG. 18. Control measurement of weight in groups of fish allocated to different vessels on Day 1. (Log 10—transformed data).

FIG. 19. Overview of the growth of the fish per diet treatment throughout the various phases of the experiment.

FIG. 20. Overview of the development of fitness factor and weight from Days 36-78.

FIG. 21. Lactate (log 10) after post-molt growth phase (D78).

FIG. 22. Weight after diet—post molt in saline phase, as well as free/total Neu5Ac in skin mucus layer.

FIG. 23. Frequency distribution for the number of lice per fish (all individuals; N=44) FIG. 24. Frequency distributions for infestation of lice (log 2 of number) for fish on control and SA diets.

FIG. 25. Lice infestation versus condition factor.

FIG. 26. Distribution of fitness factor in post smolts with an infestation of lice, according to diet.

FIG. 27. Plasma cortisol versus intensity of lice infestation.

FIG. 28. Change in condition factor from D78 to D94 (lice infestation), all fish combined.

FIG. 29. Fitness factor in the diet groups after lice infestation.

FIG. 30. Changes in mucus cell size—and density on skin and in gills after lice infestation.

FIG. 31. Dynamics in sialic acids before (D78) and after (D94) exposure to lice.

FIG. 32. Comparison of sialic acid response in skin during lice infestation, against ILAV and before infection.

FIG. 33. Frequency distributions of ILA-positive fish with/without SA diet.

FIG. 34. Levels of free Neu5Ac in fish with/without ILA infection, after diet.

FIG. 35. Course of survival—ISAV infection trial.

FIG. 36. Weight and condition factor at the end of the ILAV experiment, both healthy and infected fish.

FIG. 37. Observed weights and variation for fish with ILAV or unknown infection status (Day 107).

### DETAILED DESCRIPTION OF THE INVENTION

The present invention relates to the use of compositions comprising sialic acid to improve the health of fish in aquaculture, and in particular to inhibit or treat viral infections in fish, improve growth, and to inhibit or treat infestation of fish with sea lice.

Sialic acid is the generic name for N- or O-substituted derivatives of neuraminic acid, a monosaccharide with nine carbon atoms. The substances were first described in saliva, hence its name after the Greek word saliva: sialon. Sialic acids are found naturally in the end of the dense and complex braid of sugar molecules, proteins and lipids on cell surfaces and on many soluble proteins. Molecular, cellular and genetic studies show that sialic acids participate in the control of cell and cell matrix interactions, intermolecular interactions on cell surfaces and in interactions with other molecules in the cell's immediate extracellular environment 1.

Sialic acids are a family of nine carbon keto-aldononulosonic acids presented at the terminal ends of glycans on cellular membranes. They are abundantly displayed on the surfaces of vertebrate cells, and particularly on all mucosal surfaces. N-acetyl neuraminic acid (Neu5Ac) is the most common form in mammalian cells.

Neu5Ac can be transformed into Neu5Gc with the enzyme CMAH (cytidine monophosphate—N-acetylneuraminic acid hydroxylase). This enzyme is not found in humans due to one deletion in the gene encoding it. One assumes therefore that man has in prehistoric times lost the functionality of this gene and thus the ability to transform NeuAc into Neu5Gc. Unlike animals and some others organisms, humans can only synthesize Neu5Ac.

The two most common sialic acids are N-acetyl neuraminic acid (Neu5Ac) and N-glycolylneuraminic acid (Neu5Gc). Carbon atom No. 5 in Neu5AC (square) can be enzymatically modified into one N-acetyl group and further hydroxylated to form Neu5Gc (circle). Hydroxyl group on several carbon atoms (C4, C7; C8 and C9) may further be modified, for example with O-acetyl, 0-sulfate, O-lactyl, O-methyl and O-phosphate groups. The sialic acids are attached to carbohydrate chains on glycoproteins and glycolipids via different glucoside bonds. The most common bonds are α2,3 binding to the galactose moiety, α2,6 binding to galactose or to the N-acetylgalactosamine moiety, and α2.8 binding to another sialic acid moiety on a glycan. Sialic acids cleave the airway epithelium—and can act as receptors for viruses. Newer research with glycan microarrays and other sophisticated methods shows the complexity of the interactions between sialic acid—containing receptors on cell surfaces and viruses proteins. This provides opportunities for studies of how viruses adhere to cell surfaces in airway epithelium (4).

According to Schalcter (2017)(5) Neu5Ac is rapidly absorbed after ingestion. It is also quickly excreted via urine. The highest concentration of sialic acids is found in saliva, urine and human brain.

The structure of Neu5Ac, features four protruding functional groups (carboxylate, hydroxyl, N-acetyl and glycerol functions). This large number of functional groups enables sialic acids to participate in a number of hydrogen bonds, salt bridges and non-polar interactions at the same time. Since sialic acid is typically located at the terminus of a glycan, its binding sites are easily accessible for interactions. A large number of viruses, including many serious human pathogens (for example human Influenza A, B and C, Coxsackievirus A24 variant and enterovirus 70, human JC and BK polyomaviruses, Rotaviruses) use sialic acid in sialylated oligosaccharides for cell attachment. Neu et al., Viruses and Sialic Acids: Rules of Engagement. Curr Opin Struct Biol., (2011) 21(5), 610-618).

The present inventors have made surprising findings with respect to the use of dietary rations supplemented with sialic acid on fish and other aquatic animals. The results are provided in the Examples and Figures and may be summarized as follows.

Bioavailability and dosage level. Human studies have indicated poor bioavailability of dietary free Neu5Ac, showing rapid absorption (minutes to hours) but also rapid and relatively complete excretion of supplemented Neu5Ac via urine. Such studies have generally involved much lower relative doses (Neu5Ac: body weight) than applied in the present trial. Current publications describe the fate (routing and metabolism) of orally administered free Neu5Ac in humans as poorly understood. Only in 2021 was de novo human synthesis of KDN published, hypothesized to be an adaptation to tackle high mannose levels (conversion of mannose to KDN followed by urinary excretion). That notwithstanding, available scientific studies on humans so far indicate that free Neu5Ac is a relatively ineffective supplement in humans—as opposed to supplementation with conjugated forms. No studies have been published on bioavailability of dietary free Neu5Ac in salmon (or in fish). The present inventors have made the following findings related to bioavailability of sialic acid:
Free Neu5Ac is bioavailable to the fish.
Dietary free Neu5Ac is transported to and deposited in skin mucous, easily detectable as elevated free Neu5Ac levels, findings that are not expected if the supplement is excreted renally without becoming distributionally and metabolically available.
Dietary free Neu5Ac is also incorporated in metabolism/glycosylation, as evidenced from increased levels of total Neu5Ac in mucous in fish on Neu5Ac-diet (both skin and gut mucous).
The physiological reference level of required Neu5Ac (total Neu5Ac: total weight) in salmon tissue might be as low as 0.057 weight-%. Not considering the, as of yet unquantified, effect of bioavailability (absorption), the Neu5Ac-concentration in the trial feed (1%) was 17.5 times that ratio, which may suggest that significant excess dose was given. In the original studies where negative regulation (feedback control) of Neu5Ac synthesis in vertebrates was discovered, 100% negative regulation occurred at CMP-Neu5Ac concentrations of 60 μg/g. Such levels were observed in gut mucous juvenile fish right before the trial (Neu5Ac-diet) started, and again during the trial in gut mucous in smolt and post smolt, while then at the same time KDN levels in gut mucous increased, highly significantly, compared with the control group.
Elevated levels of free and total KDN in gut mucous in smolt and post smolt, in response to a high dose of Neu5Ac, is an unexpected finding. Neu5Ac/Neu5Gc on one hand, and KDN on the other, are considered to have separate syntheses, and a Neu5Ac-KDN interaction has not been previously published. This interaction could be interpreted as (1) a detox response, akin to KDN synthesis in humans, through synthetic mechanisms that are as of yet undescribed, or (2) less likely, some unknown process in the gut microbiome. In any event, fish on Neu5Ac-diet with the highest levels of free and total KDN did not benefit from the high levels of KDN, rather the highest KDN levels were associated with somewhat reduced growth (within group; still better growth than the control group), as evidenced by correlation analysis; again this is indicative of the dose having exceeded some optimum by a considerable factor.
Both time-stratified ANOVAs and multivariable analysis using a neural model indicated (1) significant life-history variation in the benefits of a high dose of Neu5Ac (weakly negative effect in freshwater phase and right after smoltifying, turning to a significant positive effect in smolt and post smolt), and (2) rapid buildup of benefits from low physiological Neu5Ac concentrations, followed by a plateau, as measured against effect on condition factor.

These findings give evidence for (1) the optimal dose varies from freshwater to saltwater life stage (little requirement until smoltifying process begins) and (2) required optimum doses that are significantly below that which was administered (in the range of 0.02 to 0.50%, and most preferably 0.07-0.25% of feed weight).

Fish with lower stress levels. A 25% lower lactate level was measured in healthy post smolt undergoing normal growth without disease challenges.
This indicates a more effective metabolism and a “relaxed” immune system.
Increased levels of both free and bound forms of Neu5Ac were found in both skin and gut mucus along with the reduced lactate levels and better growth.
This can give better:

Stronger/higher quality mucosal barriers. The structure of important macromolecules in salmon mucous, and the prominence of sialic acid in these is well known; such as mucins (where terminal sugars and especially sialic acid mounted on a protein backbone binds water), the dominant mucosal immunoglobulin Sialyl-Tn that is a conjugate of Neu5Ac, transferrin (also a Neu5Ac-siylated molecule in salmon), and other as well as other conjugates of relevance to mucosal defense activity. Previous studies have shown that the major genetic response of cells from salmon skin during challenges with ectoparasites, such as lice, is upregulation of the CMP gene that is crucial for incorporation of sialic acid in conjugates. As such, a more immune-effective mucous can be inferred from the significant findings for healthy post smolt on Neu5Ac-diet of
Increased levels of both free and total Neu5Ac in skin as well as gut mucous;
Elevated levels of free Neu5Gc in gut mucous (convertible from Neu5Ac via CMAH);
Reduced probability of successful virus infection (viruses with known sialic binding affinities for attachment factors as well as, in several cases, such as ISAV, RBDs targeting sialylated cell entry factors);
Reduced probability of successful bacterial/amoebic infection (generic effect as well and especially several taxa with known sialic acid-binding affinities).
   These results are interpreted to be directly relevant to positive findings on reduced infection rates (ISAV), improved growth after smoltifying and maintenance of better condition factor during lice infection. Based on existing literature, highly sialylated mucous is beneficial to capture both bacterial and viral sialic acid-binding pathogens; it has previously been shown that removal of sialic acids from salmon mucous destroyed its capacity to bind the bacterium Aeromonas salmonicida (causative agent of furunculosis); the generic value of well-sialylated mucous is well documented both in salmon and in vertebrate mucous as well as the vertebrate glycocalyx.

Beneficial effect on cellular level in mucosal epithelia. Healthy post smolt on Neu5Ac-diet generally had better condition factor than fish on control diet, and condition factor was shown to be the major determining factor for average mucous cell size and—density.
Neu5Ac-diet thus indirectly caused stronger mucosal epithelia (larger cells, higher cell density) via improved condition factor.
Healthy post smolt on Neu5Ac-diet showed a significant, additional positive effect on average skin-mucous cell size beyond what was explained by higher condition factor (though no effect on cell density, which our data indicate is determined by life history stage and fish size/condition factor).

Neu5Ac resulted in increased growth and weight after smoltifying. In healthy post smolt: 19% higher condition factor; 22% increased weight; 5% increased length. These results for fish on Neu5Ac-diet were also associated with 109% higher levels of free Neu5Ac and 52% higher total Neu5Ac levels in skin mucous compared to the control group. Fish on the diet with a high dose of free Neu5Ac thus generally had better growth; within that group, fish with the highest levels of free/total KDN, evidently triggered by the Neu5Ac-supplementation, had growth/condition factor that correlated negatively with KDN—this can be indicative of overdose-effects. The results suggests that while Neu5Ac clearly stimulated growth, the dose given was so high as to give some constraining effects via other metabolic mechanisms, i.e. the growth benefits might be found to be even higher when a somewhat lower dose of Neu5Ac is administered (cf. negative regulation and potential spillover of accumulating precursors into weakly growth-constraining effects)

The infection trial with salmon lice did not find evidence of reduced settlement of lice on fish on Neu5Ac-diet. However, the infection trial was not very representative of field conditions. There was basically no escape from infection: Every fish in the trial was infected, average incidence 11 copepodites per fish, whereas in field conditions, de-lousing of the entire salmon farm is mandated at an incidence of 0.5 lice per fish. That is, the average infection rate as 22 times higher than the level that would trigger mandatory de-lousing of the whole stock. However:
Fish on Neu5Ac-diet scored higher condition factor than the control group, showing that fish on Neu5Ac-diet grew better and tolerated infection better;
Both groups (Neu5Ac and control diets) showed significant reduction in condition factor when challenged with lice, but, as noted, less so for fish on Neu5Ac-diet;
The lice challenge trial was somewhat short, only 16 days, whereas the preceding growth period was much longer (58 days without disease challenge); it is likely that the significant benefits from Neu5Ac diet on cellular level in skin mucosa would prove significant also during an extended lice challenge trial.

The encouraging results on infection tolerance (as shown via condition factor) suggest that use under field conditions—with much lower infection rates and greater number of fish than in this trial—may reveal a contrast in lice infection rates in pre-presentative conditions, for fish on/without Neu5Ac-diet. The proposed mechanism is generic (mucous quality), as salmon lice is known to be attracted to certain kairomones (alpha-isophorone and other volatile organic compounds) whereas sialic acid is not known to be either a kairomone nor the opposite, for salmon lice.

The infection trial with ISAV gave strong, positive results for fish on Neu5Ac diet. Fish on Neu5Ac diet had significantly lower infection rate than the control group. The trial was cut short due to logistical constraints, at a time when mortality in the Neu5ac-group appeared to be levelling off compared to the control group; at this time (end of experiment), 40% of the sampled fish on Neu5A-diet were ISAV-positive, whereas 87% of the control group was ISAV-positive. Based on published information about sialic acid-binding viruses and bacteria, where the role of sialic acids to preclude ‘promiscuous’ binding mechanisms to sialylated attachment factors, and in many cases specific binding to sialylated entry factors (receptors), these results are proof-of-concept and transferrable to broad classes of pathogens with known affinities for sialic acid (non-enveloped icosahedral viruses such as PMCV, Alloherpesviridae; Neoparamoeba spp that causes amoebic gill disease, Aeromonas salmonica that causes furunculosis, etc.).

Reduced infection rates and more effective metabolism will result in fewer deaths. In the two most intensively cultivated salmon farming regions of western Norway, mortality in sea-based cage culture is 25-27% per production cycle. Most of this mortality is caused by handling stress and damages that occur when fish farmers perform de-lousing activities that are mandated when lice prevalence reaches a certain level, rather being due to lethal effects of salmon lice itself, and also from viral infections (pancreatic disease, infections salmon anemia virus, PMCV heart inflammatory virus). Recently, it has been shown that salmon with lice infections are more susceptible to viral infections (evidence given in the literature dealt with ISAV infection, the subject of the present viral disease challenge).
The fish is healthier when having higher levels of dietary Neu5Ac (cf. mucous quality, cellular level benefits in mucosal epithelia, more effective metabolism as reflected in better growth and reduced lactate).
Reduced infection rate of viruses and bacteria gives direct health- and survival benefits (improved growth, reduced infection rates).
Indirect survival benefits are now shown to be highly likely: Mortality induced by mechanical stress and handling is expected to be somewhat reduced since more effective metabolism has been indicated for fish on Neu5Ac-diet (in effect, PMCV-infected fish dies of oxygen stress when an inflamed heart muscle and weakened circulation system is confronted with the stress of mechanical de-lousing and sorting operations; higher growth rates, better condition factor and reduced lactate levels suggests that fish on Neu5Ac-diet would be less susceptible to oxygen-stress related death and would restitute faster).

Based on the literature, known precursors to Neu5Ac like mannosamine and glucosamine are expected stimulate biosynthesis of Neu5Ac. The resulting Neu5Ac levels from supplementing GlcNAc is expected to subject to the same negative feedback regulation as Neu5Ac itself, whereas supplementation with ManNAc bypasses the negative regulation mechanism and may permit Neu5Ac accumulation beyond the limits of negative regulation (since negative regulation constrains ManNAc synthesis). The Neu5Ac-KDN interaction found in this study points to the exciting but as yet unproven possibility of Neu5Ac-KDN cross-metabolism, i.e. the potential that mannose and fructose (possibly Man-6-P, Fru-6-P) may be useful Neu5Ac precursors. If mannose/fructose affect Neu5Ac synthesis (which may potentially be caused indirectly through reduced competition for precursors between alternative synthesis pathways during active growth and tissue differentiation after smoltifying), the cost of Neu5Ac (indirect) supplementation would be radically cut. It is also noted that Neu5Ac-metabolizing bacteria prefers glucose to Neu5Ac when glucose is available, and it is suggested that inclusion of some free glucose along with free Neu5Ac may therefore reduce bacterial metabolism (“hijacking”) of Neu5Ac and thus make this costly feed additive more effectively available for absorption by the fish.

Accordingly, provided herein are compositions comprising sialic acid for use in treating or inhibiting infection of fish, particularly aquacultured fish, by viruses and sea lice (Lepeophtheirus salmonis). In some particularly preferred embodiments, virus is a member of the family Togaviridae. In some particularly preferred embodiments, virus is a member of the family Orthomyxoviridae. In some particularly preferred embodiments, virus is a member of the family Birnaviridae. In some particularly preferred embodiments, virus is a member of the family Reoviridae. In some particularly preferred embodiments, virus is a member of the family Totiviridae. In some particularly preferred embodiments, the virus is salmon pancreas disease virus (also called salmonid alphavirus, SAV). In some particularly preferred embodiments, compositions comprising sialic acid are provided for use in treating or preventing pancreas disease in aquacultured fish. In some particularly preferred embodiments, the virus is infectious salmon anemia virus. In some particularly preferred embodiments, compositions comprising sialic acid are provided for use in treating or preventing infectious salmon anemia in aquacultured fish. In some particularly preferred embodiments, the virus is Infectious Pancreatic Necrosis Virus (IPNV). In some particularly preferred embodiments, compositions comprising sialic acid are provided for use in treating or preventing pancreatic necrosis in aquacultured fish. In some particularly preferred embodiments, the virus is Piscine Orthoreovirus (PRV). In some particularly preferred embodiments, compositions comprising sialic acid are provided for use in treating or preventing heart and skeletal muscle inflammation disease in aquacultured fish. In some particularly preferred embodiments, the virus is Piscine Myocarditis Virus (PMCV). In some particularly preferred embodiments, compositions comprising sialic acid are provided for use in treating or preventing cardiomyopathy syndrome in aquacultured fish.

In some further preferred embodiments, the present invention provides a method of improving one or more health parameters in an aquatic animal comprising administering to the aquatic animal sialic acid or a precursor thereof. In some embodiments, the present invention provides sialic acid or a precursor thereof for use in improving one or more health parameters in an aquatic animal. In some preferred embodiments, the one or more health parameters is/are selected from the group consisting of: a) prophylaxis of infection by a sialic acid-binding pathogen; b) treatment of infection by a sialic acid-binding pathogen; c) amelioration of symptoms caused by sea lice infestation; d) improvement of skin barrier function by increasing sialic acid content in skin mucous; e) improvement of gut barrier function by increasing sialic acid content in gut mucous; f) increasing KDN content in the gut mucous; g) decreasing stress levels as evidenced by a decrease in lactate levels; h) stimulation of mucous epithelia cells; i) improvement in condition factor after smoltifying; j) improvement of growth after smoltifying; and k) improvement in length after smoltifying.

In some preferred embodiments, the health parameter is prophylaxis of infection by a sialic acid-binding pathogen. In some preferred embodiments, the health parameter is treatment of infection by a sialic acid-binding pathogen. In some preferred embodiments, the health parameter is amelioration of symptoms caused by sea lice infestation. In some preferred embodiments, the health parameter is improvement of skin barrier function by increasing sialic acid content in skin mucous. In some preferred embodiments, the health parameter is improvement of gut barrier function by increasing sialic acid content in gut mucous. In some preferred embodiments, the health parameter is increasing KDN content in the gut mucous. In some preferred embodiments, the health parameter is decreasing stress levels as evidenced by a decrease in lactate levels. In some preferred embodiments, the health parameter is stimulation of mucous epithelia cells. In some preferred embodiments, the health parameter is improvement in condition factor after smoltifying. In some preferred embodiments, the health parameter is improvement of growth after smoltifying. In some preferred embodiments, the health parameter is improvement in length after smoltifying.

In some preferred embodiments, two or more of health parameters a) to k) are improved. In some preferred embodiments, three or more of health parameters a) to k) are improved. In some preferred embodiments, four or more of health parameters a) to k) are improved. In some preferred embodiments, five or more of health parameters a) to k) are improved. In some preferred embodiments, six or more of health parameters a) to k) are improved. In some preferred embodiments, seven or more of health parameters a) to k) are improved. In some preferred embodiments, eight or more of health parameters a) to k) are improved. In some preferred embodiments, nine or more of health parameters a) to k) are improved. In some preferred embodiments, ten or more of health parameters a) to k) are improved.

In some embodiments, the sialic acid (e.g., NANA or NGNA) or precursor thereof is provided in a fish feed at a weight percent (w/w; weight of sialic acid/total weight of the feed) of from 0.010% to 2.0% of the feed. In some embodiments, the sialic acid (e.g., NANA or NGNA) or precursor thereof is provided in a fish feed at a weight percent (w/w) of from 0.01% to 1.0% of the feed. In some embodiments, the sialic acid (e.g., NANA or NGNA) or precursor thereof is provided in a fish feed at a weight percent (w/w) of from 0.02% to 1.0% of the feed. In some embodiments, the sialic acid (e.g., NANA or NGNA) or precursor thereof is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

The use of a variety of sialic acids is contemplated. Indeed, the present invention is not limited to the use of any particular sialic acid. In some embodiments, the sialic acids or sialic acid precursors have a purity selected from the group consisting of greater than 90%, 95%, 99%, and 99.5% pure. In some preferred embodiments, the sialic acid or sialic acid precursor is selected from the group consisting of n-acetylneuraminic acid (NANA), n-glycolylneuraminic acid (NGNA), N-Acetyl-D-mannosamine, and combinations thereof. In other preferred embodiments, the composition may comprise one or more of the following sialic acids: Neuraminic acid, 5-N-Acetyl-4-O-acetyl-neuraminic acid, 5-N-Acetyl-7-O-acetyl-neuraminic acid, 5-N-Acetyl-8-O-acetyl-neuraminic acid, 5-N-Acetyl-9-O-acetyl-neuraminic acid, 5-N-Acetyl-4,9-di-O-acetyl-neuraminic acid, 5-N-Acetyl-7,9-di-O-acetyl-neuraminic acid, 5-N-Acetyl-8,9-di-O-acetyl-neuraminic acid, 5-N-Acetyl-7,8,9-tri-O-acetyl-neuraminic acid, 5-N-Acetyl-9-O-L-lactyl-acetyl-neuraminic acid, 5-N-Acetyl-4-O-acetyl-9-O-lactyl-acetyl-neuraminic acid, 5-N-Acetyl-8-O-methyl-neuraminic acid, 5-N-Acetyl-9-O-acetyl-8-O-methyl-neuraminic acid, 5-N-Acetyl-8-O-sulpho-neuraminic acid, 5-N-Acetyl-9-O-phosphoro-neuraminic acid, 5-N-Acetyl-2-deoxy-2,3-didehydro-neuraminic acid, 5-N-Acetyl-9-O-acetyl-2-deoxy-2,3-didehydro-neuraminic acid, 5-N-Acetyl-2-deoxy-2,3-didehydro-9-O-lactyl-neuraminic acid, 5-N-Acetyl-2,7-anhydro-neuraminic acid, 4-O-Acetyl-5-N-glycolyl-neuraminic acid, 7-O-Acetyl-5-N-glycolyl-neuraminic acid, 8-O-Acetyl-5-N-glycolyl-neuraminic acid, 9-O-Acetyl-5-N-glycolyl-neuraminic acid, 7,9-Di-O-acetyl-5-N-glycolyl-neuraminic acid, 8,9-Di-O-acetyl-5-N-glycolyl-neuraminic acid, 7,8,9-Tri-O-acetyl-5-N-glycolyl-neuraminic acid, 5-N-glycolyl-9-O-lactyl-neuraminic acid, 5-N-glycolyl-8-O-methyl-neuraminic acid, 9-O-Acetyl-5-N-glycolyl-8-O-methyl-neuraminic acid, 7,9-di-O-Acetyl-5-N-glycolyl-8-O-methyl-neuraminic acid, 5-N-glycolyl-8-O-sulpho-neuraminic acid, N-(0-acetyl)glycolylneuraminic acid, N-(0-Methyl)glycolylneuraminic acid, 2-Deoxy-2,3-didehydro-5-N-glycolyl-neuraminic acid, 9-O-Acetyl-2-deoxy-2,3-didehydo-5-N-glycolyl-neuraminic acid, 2-Deoxy-2,3-didehydro-5-N-glycolyl-9-O-lactyl-neuraminic acid, 2-Deoxy-2,3-didehydro-5-N-glycolyl-8-O-methyl-neuraminic acid, 2,7-Anhydro-5-N-glycolyl-neuraminic acid, 2,7-Anhydro-5-N-glycolyl-8-O-methyl-neuraminic acid, 2-Keto-3-deoxynononic acid, and 9-O-Acetyl-2-keto-3-deoxynononic acid.

In some particularly preferred embodiments, the sialic acid is NANA (Neu5Ac). In some particularly preferred embodiments, the sialic acid is NGNA. In some particularly preferred embodiments, the sialic acid is neuraminic acid. In some particularly preferred embodiments, the sialic acid precursor is N-Acetyl-D-mannosamine (ManNAc), which serves as a precursor for NANA.

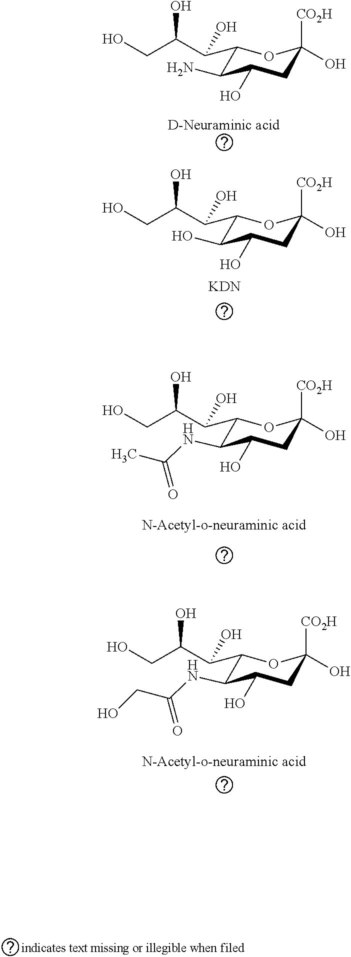

Sialic acid (pr precursor) compositions of the present invention may be delivered in any suitable format. In some embodiments, the sialic acid is preferably about greater than 90%, 95%, 99% or 99.9% pure. In some embodiments, the sialic acid is HPLC purified. For example, NANA can be purchased commercially from, for example, Sigma Chemical Company, St. Louis, MO or Chr. Hansen Holding A/S.

In some embodiments, the present invention provides methods of treating, alleviating, ameliorating, or inhibiting infection of aquacultured fish (e.g., salmon) by pancreas disease virus, or reducing symptoms (e.g., pancreas disease) or outbreaks associated with infection of aquacultured salmon by pancreas disease virus, comprising administering a composition comprising an effective concentration of sialic acid. In some embodiments, the present invention provides methods of treating, alleviating, ameliorating, or inhibiting infection of aquacultured salmon by pancreas disease virus, or reducing symptoms or outbreaks associated with infection of aquacultured salmon by pancreas disease virus, comprising administering a composition comprising an effective concentration of NANA. In some embodiments, the present invention provides methods of treating, alleviating, ameliorating, or inhibiting infection of aquacultured salmon by pancreas disease virus, or reducing symptoms or outbreaks associated with infection of aquacultured salmon by pancreas disease virus, comprising administering a composition comprising an effective concentration of NGNA. In some embodiments, the present invention provides methods of treating, alleviating, ameliorating, or inhibiting infection of aquacultured salmon by pancreas disease virus, or reducing symptoms or outbreaks associated with infection of aquacultured salmon by pancreas disease virus, comprising administering a composition comprising an effective concentration of combination of NANA and NGNA. In some embodiments, the sialic acid (e.g., NANA or NGNA) is provided in a fish feed at a weight percent (w/w; weight of sialic acid/total weight of the feed) of from 0.010% to 2.0% of the feed. In some embodiments, the sialic acid (e.g., NANA or NGNA) is provided in a fish feed at a weight percent (w/w) of from 0.01% to 1.0% of the feed. In some embodiments, the sialic acid (e.g., NANA or NGNA) is provided in a fish feed at a weight percent (w/w) of from 0.02% to 1.0% of the feed. In some embodiments, the sialic acid (e.g., NANA or NGNA) is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

In some embodiments, the present invention provides methods of treating, alleviating, ameliorating, or inhibiting infection of aquacultured fish (e.g., salmon) by infectious salmon anemia virus (ISAV), or reducing symptoms or outbreaks associated with infection of aquacultured salmon by ISAV, comprising administering a composition comprising an effective concentration of sialic acid. In some embodiments, the present invention provides methods of treating, alleviating, ameliorating, or inhibiting infection of aquacultured salmon by ISAV, or reducing symptoms or outbreaks associated with infection of aquacultured salmon by ISAV, comprising administering a composition comprising an effective concentration of NANA. In some embodiments, the present invention provides methods of treating, alleviating, ameliorating, or inhibiting infection of aquacultured salmon by ISAV, or reducing symptoms or outbreaks associated with infection of aquacultured salmon by ISAV, comprising administering a composition comprising an effective concentration of NGNA. In some embodiments, the present invention provides methods of treating, alleviating, ameliorating, or inhibiting infection of aquacultured salmon by ISAV, or reducing symptoms or outbreaks associated with infection of aquacultured salmon by ISAV, comprising administering a composition comprising an effective concentration of combination of NANA and NGNA. In some embodiments, the sialic acid (e.g., NANA or NGNA) is provided in a fish feed at a weight percent (w/w; weight of sialic acid/total weight of the feed) of from 0.01% to 2.0% of the feed. In some embodiments, the sialic acid (e.g., NANA or NGNA) is provided in a fish feed at a weight percent (w/w) of from 0.010% to 1.0% of the feed. In some embodiments, the sialic acid (e.g., NANA or NGNA) is provided in a fish feed at a weight percent (w/w) of from 0.02% to 1.0% of the feed. In some embodiments, the sialic acid (e.g., NANA or NGNA) is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

In some embodiments, the present invention provides methods of treating, alleviating, ameliorating, or inhibiting infection of aquacultured fish (e.g., salmon) by Lepeophtheirus salmonis (sea lice), or reducing symptoms or outbreaks associated with infection of aquacultured salmon by sea lice, comprising administering a composition comprising an effective concentration of sialic acid. In some embodiments, the present invention provides methods of treating, alleviating, ameliorating, or inhibiting infection of aquacultured salmon by sea lice, or reducing symptoms or outbreaks associated with infection of aquacultured salmon by sea lice, comprising administering a composition comprising an effective concentration of NANA. In some embodiments, the present invention provides methods of treating, alleviating, ameliorating, or inhibiting infection of aquacultured salmon by sea lice, or reducing symptoms or outbreaks associated with infection of aquacultured salmon by sea lice, comprising administering a composition comprising an effective concentration of NGNA. In some embodiments, the present invention provides methods of treating, alleviating, ameliorating, or inhibiting infection of aquacultured salmon by sea lice, or reducing symptoms or outbreaks associated with infection of aquacultured salmon by sea lice, comprising administering a composition comprising an effective concentration of combination of NANA and NGNA. In some embodiments, the sialic acid (e.g., NANA or NGNA) is provided in a fish feed at a weight percent (w/w; weight of sialic acid/total weight of the feed) of from 0.01% to 2.0% of the feed. In some embodiments, the sialic acid (e.g., NANA or NGNA) is provided in a fish feed at a weight percent (w/w) of from 0.01% to 1.0% of the feed. In some embodiments, the sialic acid (e.g., NANA or NGNA) is provided in a fish feed at a weight percent (w/w) of from 0.02% to 1.0% of the feed. In some embodiments, the sialic acid (e.g., NANA or NGNA) is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

In some embodiments, the present invention provides methods of treating, alleviating, ameliorating, or inhibiting infection of aquacultured fish (e.g., salmon) by Infectious Pancreatic Necrosis Virus (IPNV), or reducing symptoms (e.g., pancreatic necrosis) or outbreaks associated with infection of aquacultured salmon by IPVN, comprising administering a composition comprising an effective concentration of sialic acid. In some embodiments, the present invention provides methods of treating, alleviating, ameliorating, or inhibiting infection of aquacultured salmon by IPVN, or reducing symptoms or outbreaks associated with infection of aquacultured salmon by IPVN, comprising administering a composition comprising an effective concentration of NANA. In some embodiments, the present invention provides methods of treating, alleviating, ameliorating, or inhibiting infection of aquacultured salmon by IPVN, or reducing symptoms or outbreaks associated with infection of aquacultured salmon by IPVN, comprising administering a composition comprising an effective concentration of NGNA. In some embodiments, the present invention provides methods of treating, alleviating, ameliorating, or inhibiting infection of aquacultured salmon by IPVN, or reducing symptoms or outbreaks associated with infection of aquacultured salmon by IPVN, comprising administering a composition comprising an effective concentration of combination of NANA and NGNA. In some embodiments, the sialic acid (e.g., NANA or NGNA) is provided in a fish feed at a weight percent (w/w; weight of sialic acid/total weight of the feed) of from 0.01% to 2.0% of the feed. In some embodiments, the sialic acid (e.g., NANA or NGNA) is provided in a fish feed at a weight percent (w/w) of from 0.01% to 1.0% of the feed. In some embodiments, the sialic acid (e.g., NANA or NGNA) is provided in a fish feed at a weight percent (w/w) of from 0.02% to 1.0% of the feed. In some embodiments, the sialic acid (e.g., NANA or NGNA) is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

In some embodiments, the present invention provides methods of treating, alleviating, ameliorating, or inhibiting infection of aquacultured fish (e.g., salmon) by Piscine Orthoreovirus (PRV), or reducing symptoms (e.g., heart and skeletal muscle inflammation) or outbreaks associated with infection of aquacultured salmon by PRV, comprising administering a composition comprising an effective concentration of sialic acid. In some embodiments, the present invention provides methods of treating, alleviating, ameliorating, or inhibiting infection of aquacultured salmon by PRV, or reducing symptoms or outbreaks associated with infection of aquacultured salmon by PRV, comprising administering a composition comprising an effective concentration of NANA. In some embodiments, the present invention provides methods of treating, alleviating, ameliorating, or inhibiting infection of aquacultured salmon by PRV, or reducing symptoms or outbreaks associated with infection of aquacultured salmon by PRV, comprising administering a composition comprising an effective concentration of NGNA. In some embodiments, the present invention provides methods of treating, alleviating, ameliorating, or inhibiting infection of aquacultured salmon by PRV, or reducing symptoms or outbreaks associated with infection of aquacultured salmon by PRV, comprising administering a composition comprising an effective concentration of combination of NANA and NGNA. In some embodiments, the sialic acid (e.g., NANA or NGNA) is provided in a fish feed at a weight percent (w/w; weight of sialic acid/total weight of the feed) of from 0.01% to 2.0% of the feed. In some embodiments, the sialic acid (e.g., NANA or NGNA) is provided in a fish feed at a weight percent (w/w) of from 0.01% to 1.0% of the feed. In some embodiments, the sialic acid (e.g., NANA or NGNA) is provided in a fish feed at a weight percent (w/w) of from 0.02% to 1.0% of the feed. In some embodiments, the sialic acid (e.g., NANA or NGNA) is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

In some embodiments, the present invention provides methods of treating, alleviating, ameliorating, or inhibiting infection of aquacultured fish (e.g., salmon) by Piscine Myocarditis Virus (PMV), or reducing symptoms (e.g., cardiomyopathy syndrome) or outbreaks associated with infection of aquacultured salmon by PMV, comprising administering a composition comprising an effective concentration of sialic acid. In some embodiments, the present invention provides methods of treating, alleviating, ameliorating, or inhibiting infection of aquacultured salmon by PMV, or reducing symptoms or outbreaks associated with infection of aquacultured salmon by PMV, comprising administering a composition comprising an effective concentration of NANA. In some embodiments, the present invention provides methods of treating, alleviating, ameliorating, or inhibiting infection of aquacultured salmon by PMV, or reducing symptoms or outbreaks associated with infection of aquacultured salmon by PMV, comprising administering a composition comprising an effective concentration of NGNA. In some embodiments, the present invention provides methods of treating, alleviating, ameliorating, or inhibiting infection of aquacultured salmon by PMV, or reducing symptoms or outbreaks associated with infection of aquacultured salmon by PMV, comprising administering a composition comprising an effective concentration of combination of NANA and NGNA. In some embodiments, the sialic acid (e.g., NANA or NGNA) is provided in a fish feed at a weight percent (w/w; weight of sialic acid/total weight of the feed) of from 0.01% to 2.0% of the feed. In some embodiments, the sialic acid (e.g., NANA or NGNA) is provided in a fish feed at a weight percent (w/w) of from 0.01% to 1.0% of the feed. In some embodiments, the sialic acid (e.g., NANA or NGNA) is provided in a fish feed at a weight percent (w/w) of from 0.02% to 1.0% of the feed. In some embodiments, the sialic acid (e.g., NANA or NGNA) is provided in a fish feed at a weight percent (w/w) of from 0.02% to 0.5% of the feed. In some preferred embodiments, the fish feed comprises from 0.07% to 0.25% w/w sialic acid or precursor thereof. In some preferred embodiments, the sialic acid is a free (i.e., non-conjugated) sialic acid. In some preferred embodiments, the fish feed is supplemented with sialic acid, for example, free sialic acid, in the indicated amount.

In some further preferred embodiments, the fish feed of the present invention comprises from about 0.01% to 1.5% w/w of a purified sialic acid. As used herein, “purified” when used in relation to sialic acid refers to sialic acid concentrated in relation to a source material such as a microbial fermentation by removing sialic acid from the source material or by removing non-sialic acid compounds from the source material. In some preferred embodiments, the sialic acid is a free (i.e., non-conjugated) sialic acid. In some preferred embodiments, the fish feed is supplemented with sialic acid, for example, free sialic acid, in the indicated amount.

Typical salmon rations (feed) of the invention comprise from about 5% to 65% fish meal (including krill meal), such as 10% to 40%, e.g. 15% to 30%, such as 20% to 25% fish meal, 5% to 30% vegetable oil, such as 100% to 25%, e.g. 15% to 20% vegetable oil and 5%-15% fish oil, such as 6%-14%, e.g. 7%-13%, such as 8%-12%, e.g. 9%-11%, such as 10%-12% fish oil, expressed as % weight of component/weight of the ration (% w/w). In some embodiments, the rations have a crude protein content of from about 32% to 46%, such as 34%-44%, e.g. 36%-40%, preferably from about 36% to 42%, a crude lipid content of from about 26% to 42%, such as 30% to 40%, e.g. 32%-36%, such as 34% to 46%, preferably from about 28% to 38%. a carbohydrate (NFE) content of from about 11% to 18%, such as 12% to 17%, e.g. 14%-16%, such as 11% to 17%, preferably from about 13% to 15%, a fiber content of from about 1% to 5%, such as 2%-4.5%, e.g. 3% to 4%, such as 3.5% to 4% preferably from about 1.5% to 2.5%, an ash content of from about 4% to 7%, such as 5% to 6%, preferably about 4.5% to 6.5%, a total phosphorus content (P) of from about 0.5% to 1.4%, such as 0.7% to 1.3, e.g. 0.8% to 1.2, such as 0.9% to 1.1%, preferably about 0.6% to 1.0%, a gross energy content of from about 20 to 30 MJ/kg, e.g. 21 to 29 MJ/kg, such as 22 to 27 MJ/kg, e.g. 24 to 26 MJ/kg, such as 25 to 26 MJ/kg preferably from about 23 to 28 MJ/kg, and a digestible energy content of from about 20 to 24 MJ/kg, such as 21 to 23 MJ/kg, e.g. 22 to 23 MJ/kg. The rations may further be supplemented with vitamins and minerals.

In some preferred embodiments, the ration is a pelleted ration. The pellet sizes may preferably range from about 4 mm to 12 mm, such as 4 mm to 12 mm, 5 mm to 11 mm, 7 mm to 9 mm, 8 mm to 9 mm and most preferably from about 6 mm to 10 mm. In an embodiment the pelleted ration is selected from the group consisting of pressed fish feed, extruded fish feed and wet semi-moist feed.

In preferred embodiments, the rations are provided to an animal in need thereof (e.g., fish, preferably salmon, in an aquaculture operation) in an amount consistent with the normal daily diet for the stage of growth for the animal. For example, if the animal is an aquacultured fish, the amount of the ration provided will provide the required daily nutrients in terms of, e.g., energy, protein, fat, carbohydrates, minerals, vitamins, etc., for the fish's stage of growth or life cycle, e.g., fry, parr, first year smolt, transfer and adult salmon.

### EXAMPLES

### Example 1

Salmon were fed a control diet containing 0.02% Neu5Ac or an experimental diet of the control diet supplemented with 1% w/w Neu5Ac for 78 days, starting in the parr (freshwater) phase (days 1-50) and into the smolt (saltwater) phase (days 50-78). On day 78, the control and experimental groups were challenged with ISAV. The ISAV challenge ended at day 94.

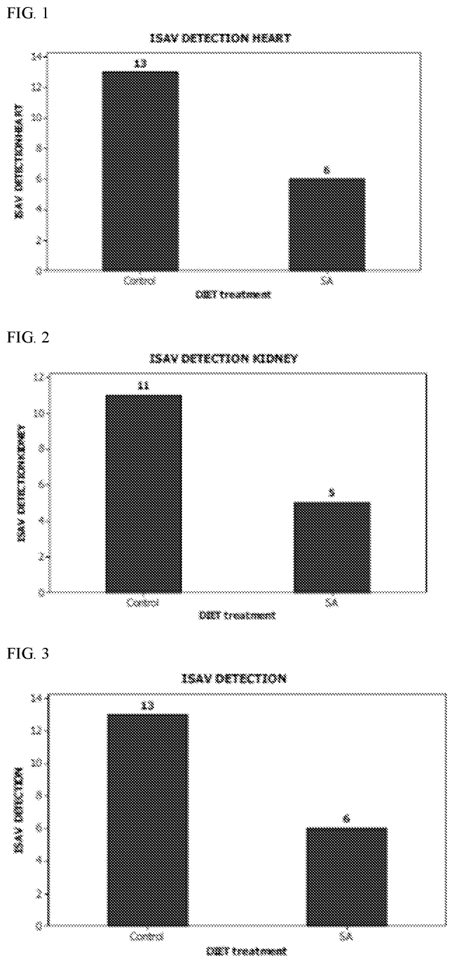

For the ISAV challenge, data was collected on accumulated mortality, weight and length, and viral load in heart and kidney. One month after exposure to ISAV, infection rates were significantly lower in fish that received the sialic acid diet. See FIG. 1 (ISAV load in heart), FIG. 2 (ISAV load in kidney) and FIG. 3 (ISAV load total). The data additionally indicated that a mortality risk reduction was emerging after 3-4 weeks.

### Example 2

An experiment was conducted to determine the mucosal health status of gills, dorsal skin and foregut of Atlantic salmon smolt and postsmolt given sialic acid (NANA) treatment at a single dose (1% in feed) or a control treatment and challenged by sea lice exposure.

Atlantic salmon were reared in the flow-through system of the ILAB facility at 12 C and were reared in freshwater through smoltification to seawater in six tanks, 3 tanks per treatment. The gills and dorsal skin of the fish (n=30 per date, 5 from each replicate tank) were individually sampled on each of the following dates. The foregut of 30 fish was sampled once, while skin samples were not collected on the same date. The samples were processed (buffered formalin fixation, paraffin embedding, sectioned and stained with PAS-AB), digitally scanned and analyzed for mucous cell density (% epithelium filled with mucous cells, A), defense (mucous) cell sizes (mucous cell area, μm², D) and defense activity (barrier status, =(1/(A:D))×1000).

The fish were transferred to two large holding tanks and challenged with salmon lice and sampled 15 days later.

The differential effects of the challenge was measurable as a reduction in cell sizes post-lice (skin). Fifteen days following challenge with salmon lice, control gills generally displayed a clear reduction in size (2 of 3 tanks) while those gills exposed to Treatment were not significantly reduced (2 of 3 tanks), suggesting an improved chronic maintenance of gill defenses through exposure to sialic acid.

### Example 3

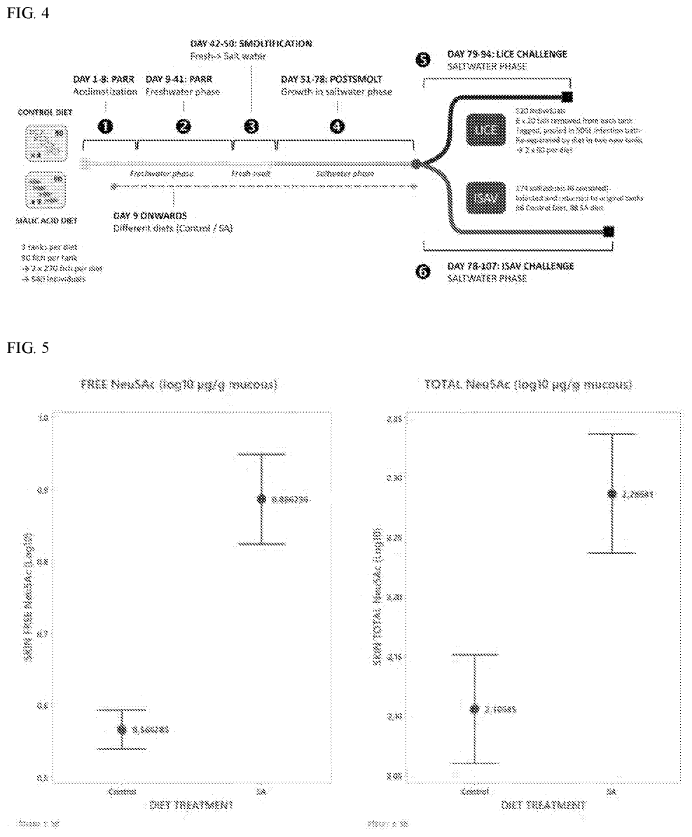

The example describes additional results obtained from feeding salmon a diet containing 1% Neu5Ac w/w. A feeding and morbidity experiment with Atlantic salmon (Salmo salar) was carried out at the Industrial Laboratory in Bergen (ILAB). The purpose of the trial was to investigate the effect of adding 1% Neu5Ac to feed on growth, certain fish health indicators, and the fish's resistance and welfare when infected with salmon lice and ILA virus, respectively. The trial included six main phases, the last two of which ran parallel with different infectious agents (FIG. 4).

The following is detailed timeline for the experiment.

Day 1-8: Acclimatization. Presmolt (approx. 40 g starting weight), freshwater phase.
Bulk weighing of all fish on day 1.
NB: All the fish received exclusively control feed during the acclimatization phase.
Day 8: First sampling.
   Day 9-41: Growth and preparation for smoltification, freshwater phase.
Different diets started (and continued) from day 9 onwards.
Light regime changed from 12:12 light/dark to 24 hours light on day 15.
Bulk weighing of fish on day 25.
Temperature increased from 12 to 14 degrees on day 28.
Smoltification starts in response to changed light regime and changed temperature.
Day 36: Second sampling.
Bulk weighing of fish on day 37.
   Day 42-50: Smoltification, transition and acclimatization to saltwater phase.
From 20 salt water from day 42, full salinity and presumed complete smoltification by day 44.
Day 50: Third sampling.
Day 51-78: Postsmolt, growth in seawater phase.
Growth on different diets, no other manipulations.
Day 78: Fourth sampling.
   Day 79-94: Infection test—salmon lice
A proportion of the fish were taken out of all test vessels on day 79; anesthetized, marked, collected in a common large vessel and infected with salmon lice.
Bulk weighing of the fish taken out for the lice test.
The experimental fish were then distributed according to original diet group, in two new vessels, for further observation.
Day 94: Fourth sampling, lice test completed.
   Day 79-107: Infection test—ILAV
Remaining fish were infected with ILAV in their original vessels on day 51.
Fish for the ILAV trial were not bulk weighed at the start of the trial.
Day 107: Fifth and final sampling, end of ILA trial.
Data was captured, with minor exceptions, on the following variables from start to finish in the experiment:
Size of the fish (length, weight);
Size and density of mucus cells on the skin and gills (as well as calculation of an index for ‘barrier activity’ from this);
Free/total concentration of Neu5Ac, Neu5Glc and KDN in skin and intestinal mucus; Plasmacortisol.

Data for histology (Quantidoc) covered skin and gills, while data on specific sialic acids (VITAS) covered skin and intestine. Histology from skin is missing for Day 36, where foregut samples were taken instead.

Cortisol in plasma was measured at all sampling events The secretion of cortisol by fish is linked to the primary (immediate, neurologically controlled) stress response, and can be used to assess the psychological aspect of the emotional part of animal welfare given that “cortisol increases in fish are linked to negative experiences and emotions. Cortisol levels are therefore highly dynamic. Catching and harvesting the fish for sampling must be expected to affect the measurements. Glucose is considered a secondary stress indicator (linked to increased heart and respiration rate), while lactate is considered an indicator of tertiary stress responses (product of anaerobic metabolism) that can provide information on stresses that can affect growth and immunity (Noble et al. 2018). Glucose and lactate were unfortunately only measured at the end of the last post-molt growth phase before infection experiments (Day 78), and are therefore not available from the end of the infection experiments with salmon lice (Day 94) or ILAV (Day 107).

Infection rate of disease (number of lice, virus infection/amount) was quantified at the end of each infection trial. Development of the infection rate along the way can only be judged from mortality data, and then only in the ILAV experiment where it is assumed that fish died of infection and not other causes.

### Results

I (a)—Bioavailability of Free Neu5Ac: Snapshot from Post-Molt Phase

To separate life history effects from a general assessment of bioavailability, the samples from the longest continuous growth phase are first analyzed without experimental manipulations. This lasted from the transition to pure salt water on day 44 until day 78.

The samples from Day 50 are not included here, as the fish, and the fish's microbiome, at that time may have been affected by the smoltification process and by recent changes in salinity in the environment.

The samples taken from Day 78 are used for the general assessment of bioavailability.

Measurements from skin—free/total Neu5Ac. Measurements of free and total Neu5Ac mucus layer in the skin are shown in FIG. 5. Free Neu5Ac in the outer mucus layer was significantly higher in postsmolts on the SA diet than on the control diet (P=0.000); the same was true for total Neu5Ac (P=0.013).

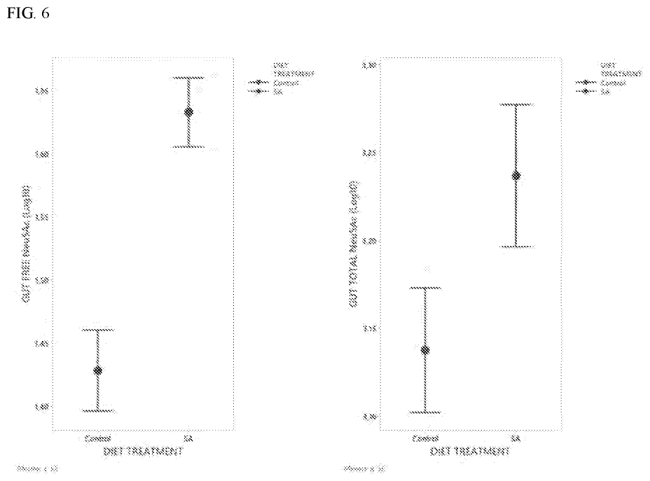

Measurements in intestine—free/total Neu5Ac. Free and total SA in the intestinal mucosa is shown in FIG. 6. Free Neu5Ac in the intestinal mucosa was significantly higher on the SA diet than on the control diet (P=0.000). Elevated level of total Neu5Ac was marginally significant (P=0.08).

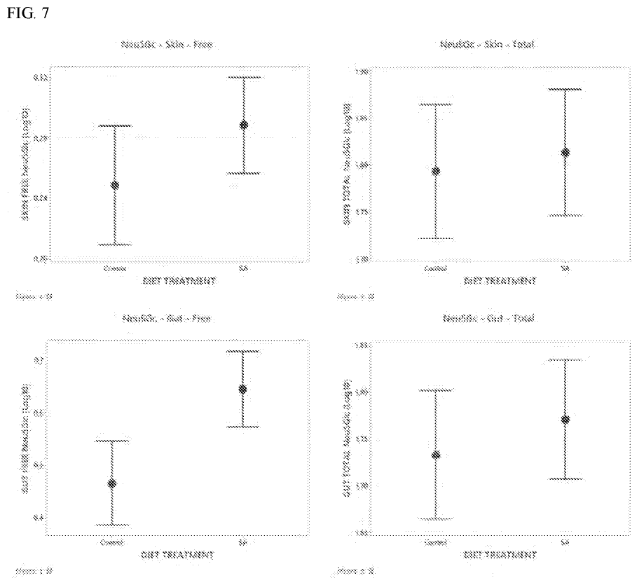

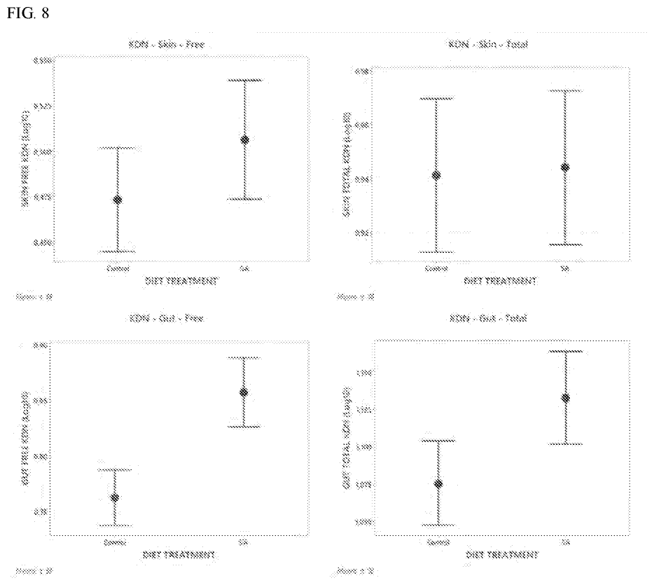

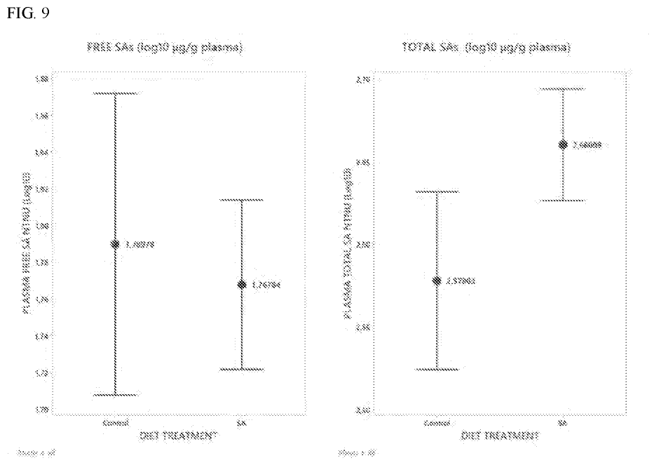

Metabolic interactions—Neu5Glc and KDN on the skin and in the gut. Specific measurements of free/total Neu5Glc in the mucous layer of the skin and intestine are shown in FIG. 7, and correspondingly for KDN in FIG. 8. Qualitatively, one sees a tendency towards higher measurements of both SA types in fish on the Neu5Ac diet, and more pronounced in the intestinal mucus. Neither Neu5Gc nor KDN showed significantly increased levels in the mucus layer on the skin. In the intestinal mucosa, on the other hand, elevated levels of free Neu5Gc were weakly significant (P=0.106), free KDN clearly significant (P=0.028) and total KDN close to significance (P=0.188).

Plasma—non-specific measurements of sialic acids. Non-specific/general measurements for all sialic acid species in plasma on Day 78 are shown in FIG. 8. Free SA (all species) in plasma was not significantly higher in postsmolt on the SA diet than on the control diet (one-way ANOVA on log 10-transformed data. DF 1, 29; F=0.05; P=0.82). Total SA (all species) in plasma was also not significantly higher in postsmolts on SA diet than on control feed (one-way ANOVA on log 10-transformed data. DF 1, 29; F=1.63; P=0.212). The clear results found for Neu5Ac, as well as for KDN, and more marginally for Neu5Ac in the specific measurements, are masked when all species sialic acids are measured with a non-specific protocol.

Conclusions: Bioavailability of free Neu5Ac in feed. The test results from the evaluation of bioavailability based on the “still image” on Day 78 are given in Table 1. Absorption and distribution of free Neu5Ac through diet has been confirmed through significantly higher values of free Neu5Ac in the mucous layer of the skin. Metabolic utilization is confirmed through significantly increased values of total Neu5Ac in the outer mucus layer. Excretion through the mucous membranes has been shown as a consequence of this. Other excretion, which would be considered a loss of Neu5Ac as a feed resource (through urine, feces) has not been assessed. Similar variations for Neu5Ac in skin and gut suggest that the significantly increased values of free and total Neu5Ac in gut also reflect both uptake and metabolism. Metabolic interactions are shown through significantly higher levels of free KDN in the intestinal mucosa, and are indicated by slightly significantly increased levels of total KDN and Neu5Gc in the intestinal mucosa. The observed effect on KDN is unexpected.

TABLE 1
ANOVA test results, SA diet~Neu5Ac, Neu5Gc and
KDN in mucus on skin and gut, Day 78 (fresh postsmolt)

| IN species | Mucous | Form | DF | F-Value | P-Value |
|---|---|---|---|---|---|
| Neu5Ac | Skin | Free | 1, 28 | 22.19 | 0.000 |
| | | Total | 1, 28 | 7.11 | 0.013 |
| | Gut | Free | 1, 26 | 23.89 | 0.000 |
| | | Total | 1, 26 | 3.31 | 0.080 |
| Neu5Gc | Skin | Free | 1, 28 | 0.61 | 0.440 |
| | | Total | 1, 28 | 0.04 | 0.839 |
| | Gut | Free | 1, 26 | 2.8 | 0.106 |
| | | Total | 1, 26 | 0.16 | 0.690 |
| KDN | Skin | Free | 1, 27 | 0.57 | 0.457 |
| | | Total | 1, 28 | 0.01 | 0.942 |
| | Gut | Free | 1, 26 | 5.41 | 0.028 |
| | | Total | 1, 26 | 1.83 | 0.188 |

### I (b) Variations in Sialic Acid Dynamics Against Diet in Different Life History Stages

The snapshot from Day 78 above, as well as the general variations in sialic acid measurements shown earlier, have shown that Neu5Ac is bioavailable and is part of metabolism. The time and life history axis can give different results in the effects of SA-enriched diet.

Neu5Ac—life history stage. In the following figures, statistically significant effects of SA diet at a given stage are indicated by ÿ. The significance level is indicated above each indicator. In each case, one-way ANOVA (‘DIET’ vs. log 10′[SA]-LEVEL′, where [SA] is the relevant free or total sialic acid; N=2×15 per test, occasionally with 1-3 censored individuals]

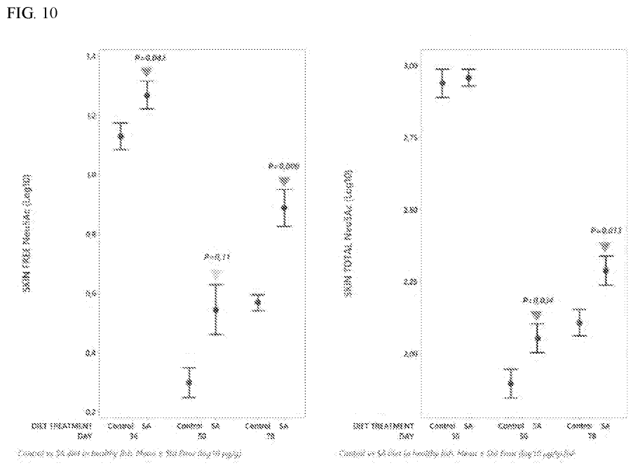

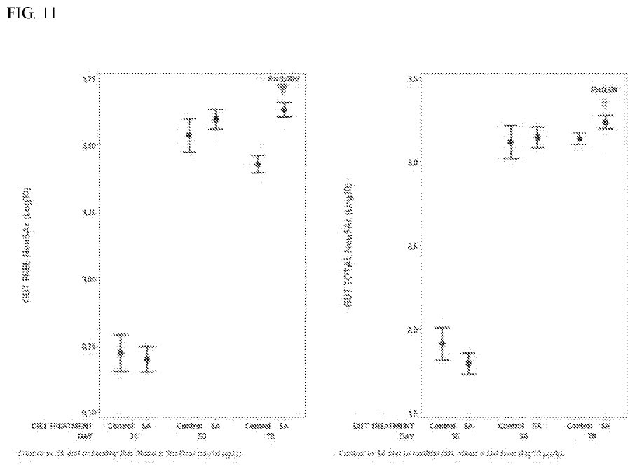

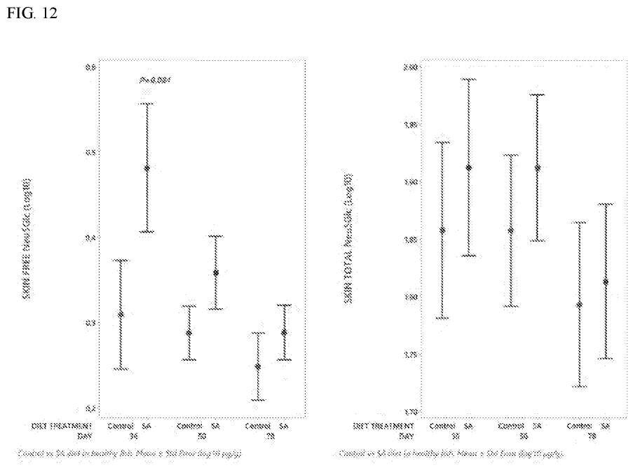

Neu5Ac in mucus layer on skin. Results for free and total Neu5Ac in mucus layer on skin are shown in FIG. 10. For free/total Neu5Ac in the mucous layer of the skin we find the following. In general across these three stages, we see that presmolt in the freshwater phase (Day 36) had far higher values of both free and total Neu5Ac in the outer mucus layer than fish during smoltification and postsmolt in the saltwater phase. Recently smoltified fish (Day 50) have significantly lower values of both free and total Neu5Ac in outer mucus layer than acclimated post smolt in saline phase (Day 78); i.e., after a decrease around smoltification, the amount of free and total Neu5Ac in the outer mucus layer increased. The increase occurred much faster in fish on the SA diet. Free Neu5Ac in mucus on the skin increased significantly in fish on the Neu5Ac diet, across all life history stages (FIG. 10, left column) (one-way ANOVA on log 10-transformed data: DF 1, 162; F=5.0; P=0.027 all stages combined). By the end of growth in sea phase (Day 78), the effect of Neu5Ac in the diet was highly significant both for free and bound forms of Neu5Ac (cf. analysis of bioavailability above). Total Neu5Ac in the outer mucus layer was significantly higher for fish on the SA diet in recent years smoltified (Day 50; P=0.034) and postsmolt in saltwater phase (Day 78; P=0.013); but not for presmolt in freshwater phase (Day 36, ANOVA on log 10-transformed data: DF 1.23: F=0.11; P=0.742).

Comparison of the measured levels on Day 50 and 78 shows that fish that received the SA diet ‘accelerated’ the build-up of both free and total Neu5Ac in the outer mucus layer after smoltification and introduction to salt water by an estimated 1 month. The levels for the control group by day 78 were already reached by fish on the SA diet around day 50. That the increase was particularly high for free Neu5Ac and to a lesser extent for total Neu5Ac after 78 days, may have relevance for dose-response considerations at a later stage.

Neu5Ac in the mucus layer of the intestines. Results for free and total Neu5Ac in mucus layer on skin are shown in FIG. 11. For Neu5Ac in intestinal mucus we find the following. Presmolt in freshwater again stand out from recently smoltified fish and postsmolt in saltwater phase, but this time with significantly lower values, both of free and total Neu5Ac in the intestinal mucus, than in the two later stages. The results in FIG. 10 and FIG. 11 indicate that there is a dramatic reversal in the ‘prioritization’ of Neu5Ac levels in skin mucus versus intestinal mucus, at the transition from press smolt in fresh water to sea water and smolt stage.

The absolute values in intestinal mucus of smolts and postsmolts for both free and total Neu5Ac were far higher than in skin mucus for the same stages (cf. FIG. 10 vs. FIG. 11). Since clear effect of SA diet has been established internally in the time periods, both in the assessment of bioavailability and in data for skin mucus, there is no reason to believe that the high values in intestinal mucus are due to late absorption, or passive accumulation. The pattern of early build-up of free/total Neu5Ac in intestinal mucus in the seawater phases must reflect an active biological process. It could either be that the fish physiologically prioritizes high levels of Neu5Ac in the intestinal mucus when transitioning to and growing in seawater, or that at this stage an intestinal flora is established where active microbial “hijacking” of Neu5Ac takes place (a known trick of commensal bacteria to avoid provoking the host's immune system. Since salmon also naturally have Neu5Glc, the microbes could potentially also “hijack” Neu5Glc for the same effect—considered in the section on Neu5Glc/intestinal mucus below).

Free Neu5Ac in feed had a significant effect on free and total Neu5Ac in intestinal mucus first cough postsmolt in saline phase (Day 78). At this time, the effect was highly significant for free Neu5Ac and weakly significant for total Neu5Ac. The total levels of Neu5Ac in intestinal mucus may appear to have stabilized in the period between Day 50-Day 78. When fish on the control diet simultaneously showed relatively low values of free Neu5Ac on Day 78 (FIG. 11), and had low values both of free and total Neu5Ac in skin mucus at the same time (FIG. 10), it may appear that the limited supply of free Neu5Ac in relation to body size and growth to cover the fish's build-up of Neu5Ac in the gut, and especially in the skin layer, has here begun to assert themselves.

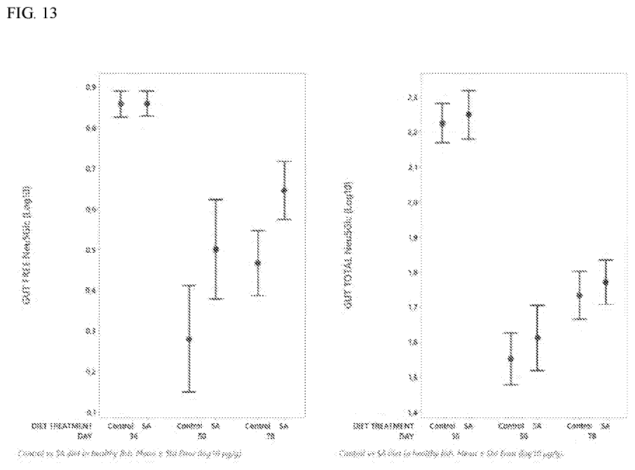

Neu5Gc—life history stage. Neu5Glc in mucus layer on skin is provided in FIG. 12. There was generally no significant effect on free or total Neu5glc in the mucous layer of skin. Qualitatively, there was a tendency towards higher values of free Neu5Glc, weakly significant in presmolt (Day 36), but decreasing with time, and not at any stage significant for total Neu5Glc (N=2×12). Referring to FIG. 13, presmolt in fresh water (Day 36) had higher values in intestinal mucus of both free (one-way ANOVA, DF 2, 72; F=18.05; P=0.000) and total Neu5Glc (DF 2, 76; F=49.25; P=0.000) than smolt and postsmolt in salt water, while measurements in smolt/postsmolt did not differ significantly from each other. SA diet had no significant effect on the levels of free/total Neu5Glc in intestinal mucus at any of the stages.

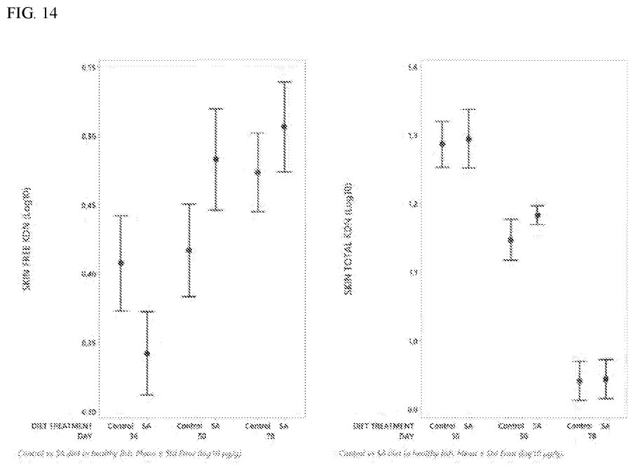

KDN—life history stage. KDN in mucus layer on skin if provided in FIG. 14. Presmolt in freshwater phase had lower levels of free KDN in the mucous layer on the skin than smolt and postsmolt in seawater (one-way ANOVA, DF 2, 80; F=5.46; P=0.006), and higher levels of total KDN, which decreased in the two later stages (DF 2, 82; F=71.49; P=0.000). There was a clear decreasing trend in total KDN in skin mucus over time, through the three stages (linear regression analysis: (log 10) Total KDN=1.581-0.00827*DAY; R2=0.63; P=0.000). SA diet had no significant effect on the levels of either free or total KDN in skin mucus.

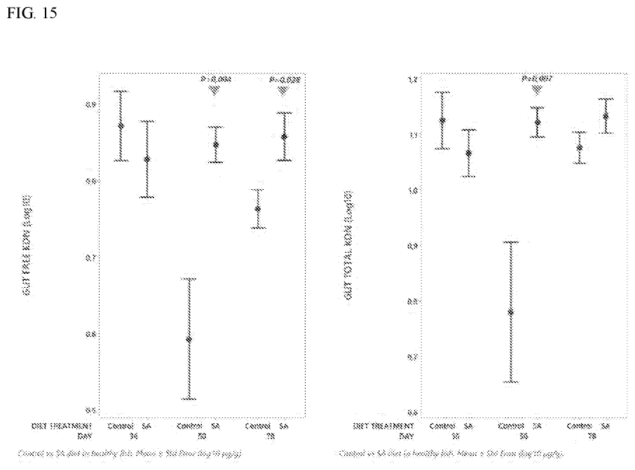

Referring to FIG. 15, recently smolted fish (Day 50) on the control diet had strikingly and significantly lower values of both free and total KDN in intestinal mucus than presmolt and postsmolt. SA diet produced significantly higher values of both free and total KDN in recently smolted fish, and higher values of free KDN in postsmolt; the latter qualitatively visible also for total KDN in postsmolt, but not significantly (P=0.188). Neu5Ac and KDN have separate synthesis. The significant effects of Neu5Ac on KDN levels found here are therefore unexpected. Such a cross-influence from Neu5Ac to KDN is not previously known.

### II—Effect on Mucosal Health in the Skin and Gut

The experiment has shown that free Neu5Ac in feed is bioavailable, both through absorption, digestion and metabolism. That free Neu5Ac in feed is taken up by the fish through digestion and distributed through plasma is documented by the fact that higher values of Neu5Ac were found both in the skin and gut. That it is included in catabolism and the formation of complex compounds is documented by the fact that significantly increased values were found for both free and total forms of Neu5Ac.

The experiment has also revealed interesting nuances in the effects of free Neu5Ac both on Neu5Ac, Neu5Glc and KDN. Observed positive correlations between free Neu5Ac added to feed, and measured values of free and total sialic acids in the mucus layers which are the salmon's most important barriers to the environment, from graphs in FIGS. 11-15 and associated statistics, are shown in Table 2.

TABLE 2

| | Significant effects of free Neu5Ac on the mucus layer on the skin and in the gut | | | | | |
|---|---|---|---|---|---|---|
| SMART | SA-art Neu5Ac (F) Neu5Ac (T) Neu5GIc (F) Neu5GIc (T) KDN (F) | | | | | KDN (T) |
| HUD | pre-melted | 0.042 | | 0.084 | | |
| | Smolt | 0.11 | 0.034 | | | |
| | Postsmolt | 0.000 | 0.013 | | | |
| GUT | pre-melted | | | | | |
| | Smolt | | | | 0.004 | 0.007 |
| | Postmolt | 0.000 | 0.08 | | 0.028 | |

The following effects were observed for the mucous layer on the skin: Increased levels of free Neu5Ac in presmolt, smolt and post smolt; No increase in total Neu5Ac in young presmolts, which had high values in initially, also on a control diet; and Increased levels of total Neu5Ac in the skin-mucous layer of smolts and postsmolts in sea phase. It is interesting that presmolt on the SA diet had high values of both free and total Neu5Ac in skin mucus regardless of the diet, and that the addition of free Neu5Ac to this led to a continued increase in free, but not total, Neu5Ac.

This may indicate that the total Neu5Ac levels were so high in small fish before smoltification, that the negative feedback regulation mentioned in the introduction kicked in. The fact that both free and total levels of Neu5Ac generally fell radically during smoltification, and then showed signs of rebuilding in postsmolt, indicates that the need for supply of Neu5Ac in the feed kicks in fully during smoltification and transition to seawater. That the total Neu5Ac levels after smoltification built up much faster in smolts and postsmolts fed the SA diet shows that the fish utilized supplemented, free Neu5Ac in the synthesis of complex compounds through a critical life history phase that is often associated with significant mortality in the salmon industry.

There were no major effects on free/total Neu5Glc, with the possible exception of presmolt in fresh water.

In the intestinal mucus, the levels were low in presmolts, but high in smolts and postsmolts both on control and SA diets.

There is a radical change and apparent reversal in the fish's expression of free/total Neu5Ac on skin versus gut, at the transition from presmolt/freshwater, to smolt and post-smelt in fresh water.

### III—Histology in Mucous Membranes on the Skin and in the Intestine

Overall pattern from parr to post smolt. No data was collected on the size and density of mucus cells on the skin of presmolts (Day 36). On the other hand, skin samples were taken for young presmolts after the acclimatization phase (Day 8), but until this time all the fish had gained weight only on the control diet. This means that there is unfortunately no data for comparing the effect of diet on the size and density of epithelial cells in the mucous membrane of the skin through the smoltification process.

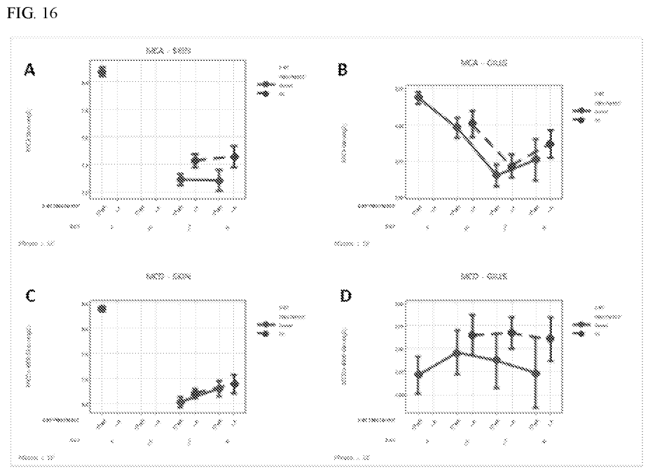

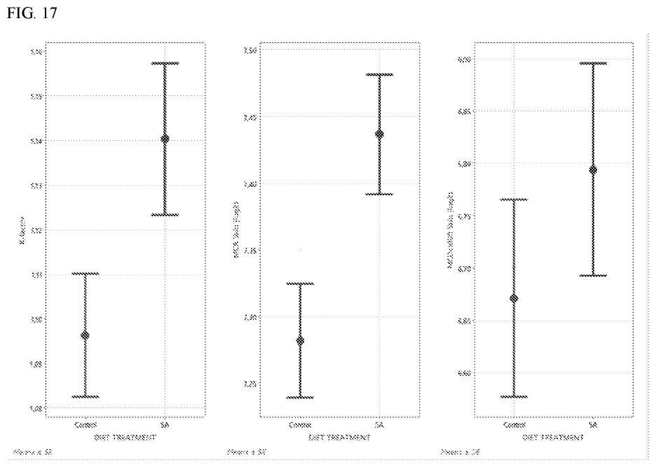

FIG. 16 shows a qualitative overview of changes in cell size and density both on skin and in gills from Day 8 (freshwater, presmolt, all fish on control diet) to Day 78 (saltwater, postsmolt). There is a sharp drop in cell size both on the skin and in the gills through smoltification, followed by an increase. The density of cells in skin follows the same pattern, a drop from the highest density in small presmolt, a minimum around smoltification, then a slightly increasing density. The density of cells in the gills shows the opposite pattern; low before smoltification, apparently a maximum around smoltification, then decline.

Postsmolt in sea phase: Mucus cell size (MCA) and density (MCD) on skin. Since both life history and fish size (allometric effects on physiology and histology) are important factors, we look in particular at separating the factors (size, diet) in the postsmolt stage before further analysis. The effect of diet on the size and density of mucus cells was analyzed for the growth period after smoltification until the experiments with lice or ILAV were to start, i.e. data from the withdrawals on Day 50 and Day 78 (FIG. 17). Postsmolt on the SA diet had significantly larger mucus cells on the skin than fish on the control diet (one-way ANOVA on log 2MCa vs. Diet; DF 1.58, F=6.24; P=0.015). Back-calculated from log 2, fish on the control diet had mucus cells in the skin with mean size MCA=155.2±1.8 ÿm2, 173 ÿm2 (mean±SD) in fish on the SA diet, an increase of 11, 5%. The apparent higher cell densities in fish on the SA diet were not significant (for MCD: DF 1.58; F=0.79; P=0.378).

However, it must be taken into account that postsmolt on the SA diet had a significantly higher fitness factor than fish on the control diet (DF 1.59; F=4.07, P=0.048), and the effect of fitness factor on both MCA and MCD has been established. The effects of diet on both mucus cell size and density were therefore assessed in regression analyzes with both fitness factor and diet as factors. When fitness factor was taken into account, the SA diet's effect on MCA was still significant (P=0.05).

The SA diet's effect on density (MCD) was not significant (P=0.993). Cell density MCD had a significantly stronger relationship with fitness factor (regression analysis; log 2MCD=6.006×K-factor; R2=99.36, P=0.000). The qualitatively visible, but not significant, effect of the SA diet on MCD is therefore an indirect effect of fish on the SA diet having a better fitness factor, which in turn correlated with MCD.

### Conclusions: Effects of SA Diet on Mucus Cells in Fresh Post Smolt in Sea Phase

We can determine for fresh post smolt in sea phase that: Post smolt fed the SA diet had a larger mean mucus cell size; Some of the effect is due to better growth, and a higher fitness factor, which affects mucus cell size; SA diet has a significant positive additional effect on mucus cell size, on top of the indirect beneficial effect via size and fitness factor. Density of mucus cells (MCD) is determined to a greater extent by size and fitness factor than size of mucus cells (MCA) is. SA diet strengthens the development of larger mucus cells after smoltification.

### IV. Effect on Growth and Condition Day 1: Weighing of Experimental Fish—Control of Satisfactory Random Distributions

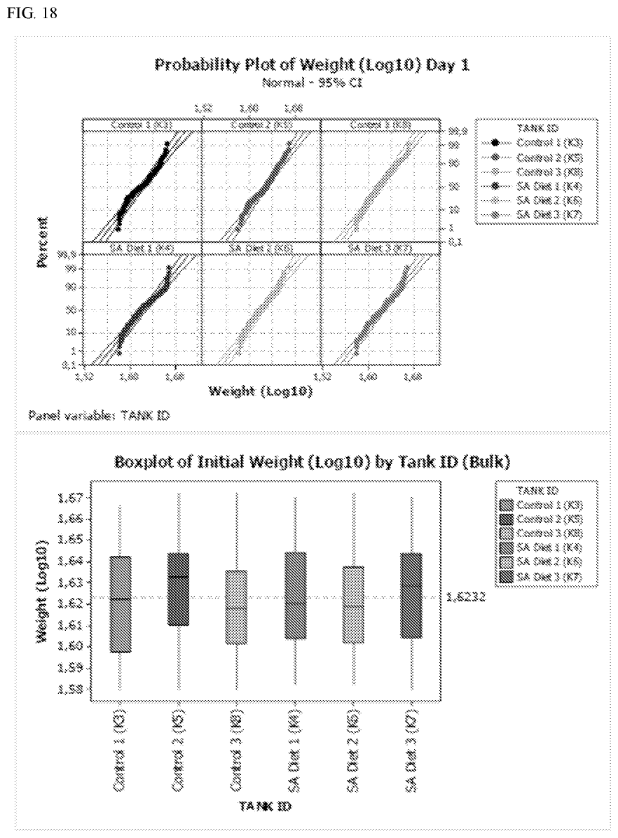

At the start of the experiment, the fish (geometric mean weight for the entire population: 42.0±1.1 grams) were distributed in different vessels. See FIG. 18. In order to prevent systematic errors, it is important that no significant differences in starting weights are formed by the random distribution. There were no significant differences in the average weight of the fish between the experimental vessels at the start of the experiment (FIG. 18; one-way ANOVA).

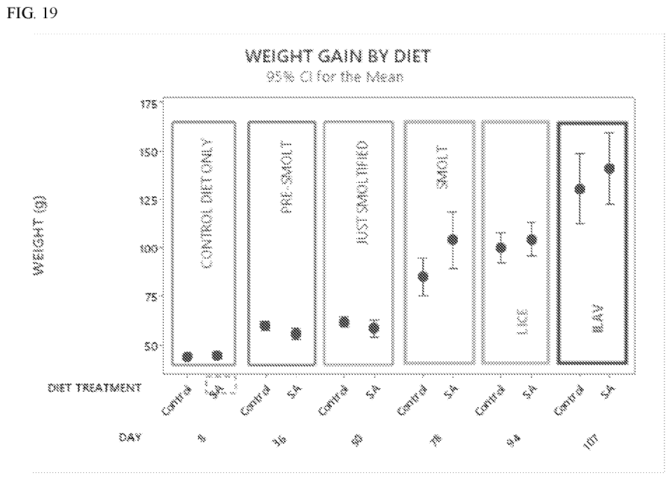

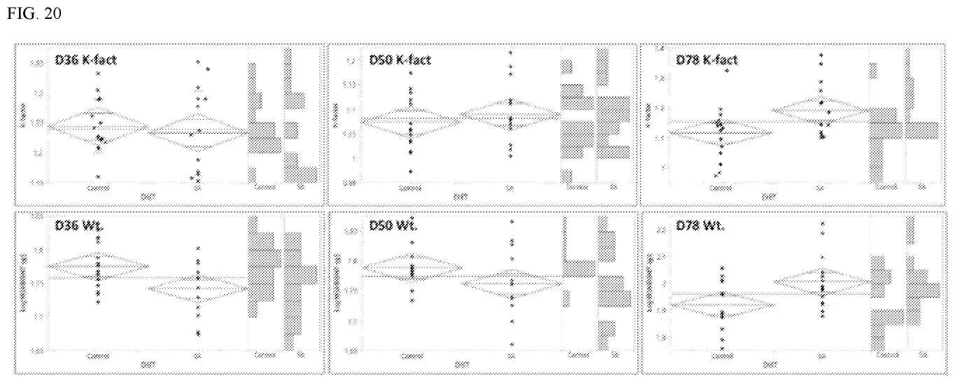

Day 8-107 (entire experiment): Overview of weight development. The test fish's final weights after the various phases of the experiment are illustrated in FIG. 19. Note that fish that were selected for infection trials with salmon lice were physically handled, VIE marked, and transferred to new vessels, while fish that were exposed to ILAV infection remained in the original vessels and were subjected to less handling. Purely visually, one can see that there is no significant separation in growth in the freshwater and smoltification phases (Day 8 up to and including Day 50). In the growth phase for acclimatized postsmolt in the saltwater phase (days 51-78), on the other hand, there is apparently a significant growth advantage among fish that received feed with added sialic acid.

Day 36: Presmolt (freshwater). At sampling on day 36, fish in the SA group weighed 7.3% less than the control group (DF=1.28; F=5.26, P=0.0295), and the fitness factor between the groups was not significantly different.

In the SA diet group, weight was negatively correlated with total Neu5Ac, total Neu5Gc and total KDN in intestinal mucus (correlation factor, significance levels, respectively −0.45/0.09; −0.59/0.021; −0.40/0, 13), i.e. strongest, and significantly, with total Neu5Gc. For measurements in skin mucus, weight was weakly negatively correlated with free Neu5Ac in the SA group (correl.coef./P: −0.45/0.1396). None of these correlations were significant in the control group, but for total Neu5Ac and total KDN in intestinal mucus, the variation with weight for these measurements went in the opposite direction.

The overviews in FIG. 4 and FIG. 13 showed that the fish (all on the control diet) had started with very high values of Neu5Ac on Day 8; by Day 36, levels of both free Neu5Gc had fallen, and Neu5Gc increased sharply, regardless of diet. This could possibly indicate that the supply of a significant dose (1% of feed weight) of extra, free Neu5Ac, which can be converted to Neu5Gc, has been an overdose for the presmolt.

Day 50: Smolt (salt water). There was no significant difference in weight between the control and SA groups in newly smoltified fish, but the weight variation in the SA group was now much greater than in the control group. Qualitatively, the fitness factor in the SA group had increased compared to the control group. The picture had changed in terms of correlations within each of the groups, between size and sialic acids. In the control group there was now a significant and strong negative correlation between weight and free Neu5Ac in the intestine (correl.coef./P: −0.86/0.0009), as well as a weaker, but significant, negative correlation between weight and total Neu5Gc in intestine (−0.469/0.0299); these connections were absent in the SA group.

Day 36-78: Development of fitness factor. As mentioned above, the SA group had weaker growth up to Day 36, no significant difference in weight by Day 50, while negative correlations between sialic acid levels and growth disappeared from the early to the later time, and there was a qualitatively better development of fitness factor. This development continued into the postsmolt growth phase and is, together with relative weight development. Seen in context with the significant changes in sialic acid dynamics, this appears to reflect a change in proportionality between the dose of Neu5Ac in the feed and the fish's physiological needs, before, via and after smoltification.

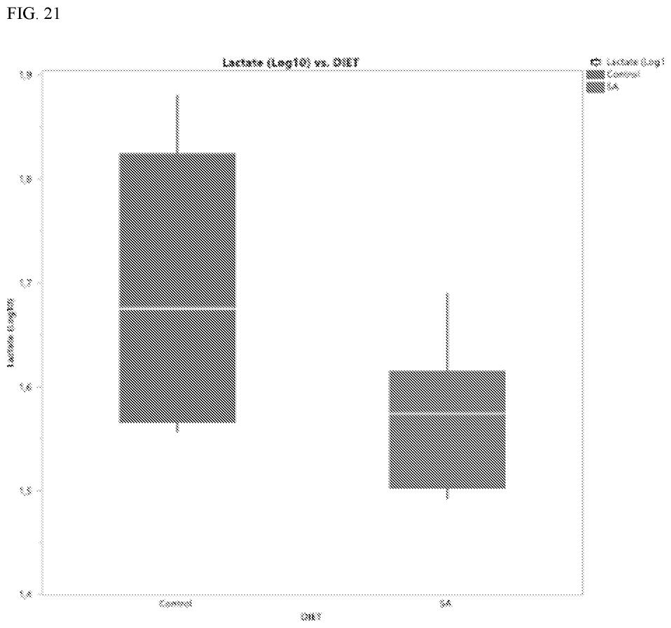

Day 78: Lactate (metabolic stress indicator). Lactate was only measured on Day 78 of the experiment, after the post-molt growth phase in seawater and before the infection experiments started (FIG. 21). Fish that were supplemented with Neu5Ac had approx. 25% lower lactate levels than control groups (t-test, DF=10, P=0.04). Lactate is the anion of lactic acid (i.e., lactic acid that has released H+ to the environment), and is naturally produced in low levels by glycolysis. During aerobic respiration, glucose is normally broken down to pyruvate, which is further included in the Krebs cycle. If the oxygen supply does not match the glucose-respiration needs, the conversion to pyruvate is delayed (“oxygen debt”) and lactate accumulates. The lower levels of lactate in fish on the SA diet therefore indicate a more efficient steady-state condition in the fish's metabolism during postsmolt growth in the sea phase.

### Day 78: Post-Molt Growth in Saltwater Phase.

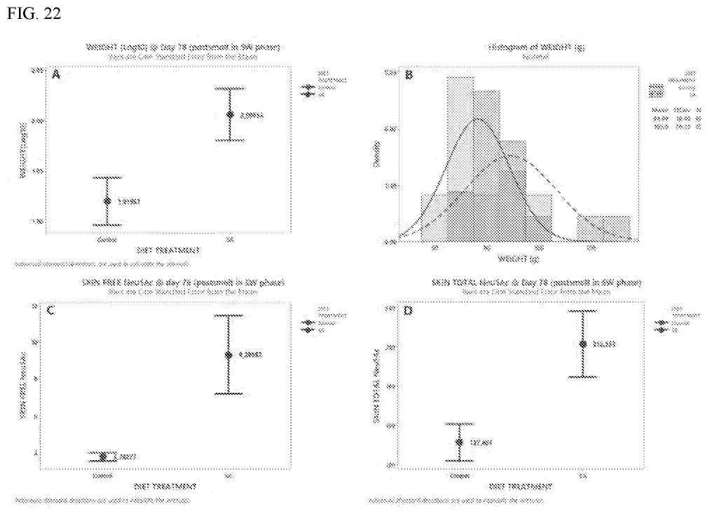

On Day 78, at the end of post-molt sea-phase growth and before sickening the fish, 31 randomly selected fish were weighed individually. The results of weighing, as well as measurement of free/total Neu5Ac in the mucous layer of the skin, are shown in FIG. 22. The post molt on the SA diet had significantly higher weight than the control group (graph A; one-way ANOVA, log 10weight vs. diet; DF 1, 29, F=6.12, P=0.019). The weight distributions were shifted to the right (B) and were log 10—transformed to calculate geometric mean weights (Table 9; geometric mean is lower than average, for both groups, and takes the distribution into account better). Postsmolt on the SA diet had 22% higher weight, 19% higher condition factor (P=0.026) and 5% greater length (P=0.048) than the control group after 78 days. As shown earlier had it also significantly higher values of free (P=0.000) and total (P=0.013) Neu5Ac in the mucous layer of the skin. The results strongly support a growth and fitness benefit of the SA diet for postsmolt.

TABLE 3
Average weight, condition factor and Neu5Ac in skin
mucus - postsmolt (Day 78) on different diets.

| Geometric cut | SA diet | Control | % Increase |
|---|---|---|---|
| Weight (g) | 101.2 | 83.1 | 22% |
| Fitness factor | 1.2 | 1.0 | 19% |
| Length (cm) | 20.4 | 19.5 | 5% |
| Fri Neu5Ac (skin) ÿg/g | 7.7 | 3.7 | 109% |
| Total Neu5Ac (skin) ÿg/g | 193.4 | 127.6 | 52% |

### V. Resistance to Salmon Lice

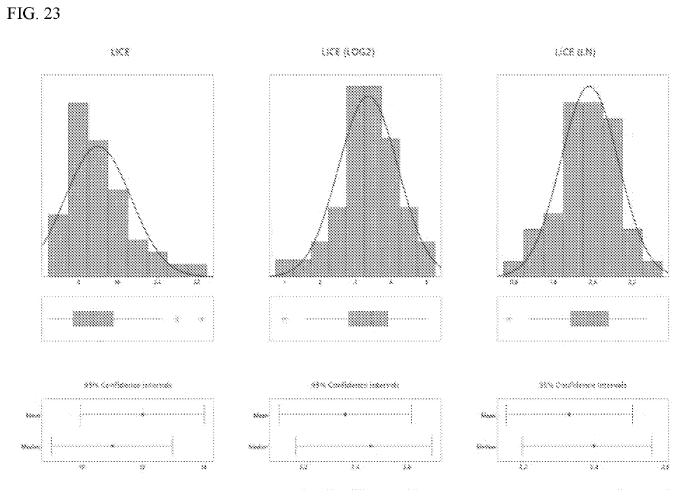

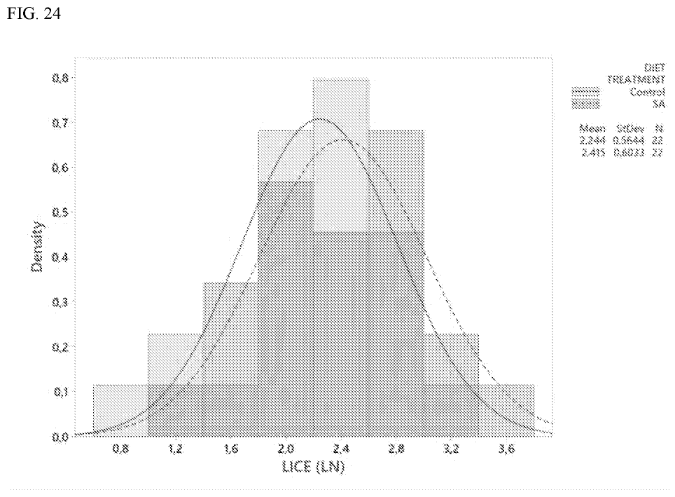

Infestation of salmon lice. At the end of the experiment with the addition of salmon lice on Day 94, the number of lice was counted for 44 fish. The frequency distribution was shifted to the right and evaluation of transformation for normalization gave the best result for natural logarithm (FIG. 23) (Anderson-Darling normality test after transformation: P=0.729, where P<0.05 would reject the assumption of normality). The frequency distributions of normalized data for infestations of lice for the two treatment groups are shown in FIG. 24. Qualitatively, one sees a tendency towards greater infestations in fish on the SA diet, which was not significant (one-way ANOVA; DF 1.42, F=0, 95, P=0.35).

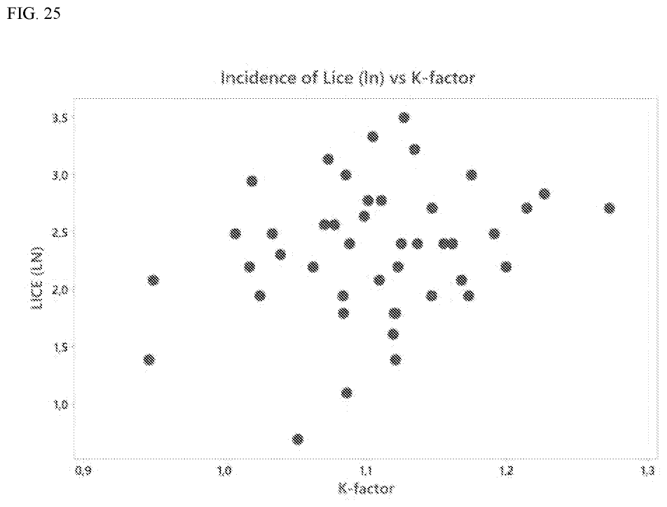

Fish on the SA diet had a significantly higher fitness factor than the control group, and associated better scores for mucus epithelium. Even a qualitative, non-significant hint in FIG. 24 that fish on the SA diet would suffer some kind of handicap linked to the diet, does not rhyme with the fact that they score better on the fitness factor and mucus health. As we have seen, i.e., in the analyzes of mucus cells, the condition of the fish is a fundamental factor that influences many other factors. It is therefore appropriate to investigate whether size/condition factor and infestation of lice have any connection. The fish's average weight SD was 102.18±18.31 g. A plot of fitness factor against infestation of lice is shown in FIG. 25.

Linear regression shows, perhaps surprisingly, a clearly significant relationship between size/condition factor and infestation of lice (model: InLus=2.1055×condition factor, R 2=94.45%, P=0.000). Why fish with a high fitness factor had a small, but significant, higher infestation of lice than fish with a lower fitness is speculation, but the connection is clear. One possible explanation is that fish with a higher fitness factor have a larger skin surface in relation to their length.

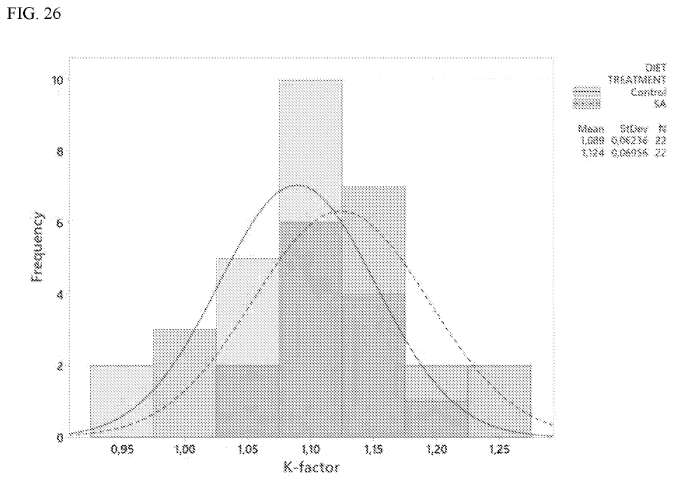

The distributions of fitness factor against diet are shown in FIG. 26. Fish on the SA diet had higher fitness factor. The higher fitness factor in the SA group thus explains the qualitative shift in lice infestation observed in FIG. 24. In a general regression model of infestation of lice against condition factor (continuous predictor) and diet (binary variable), i.e. where the variation from both condition factor and diet is treated in the same model, condition factor retains high significance (R2=94.36, P=0.000), while the significance of SA diet falls away completely (P=0.565).

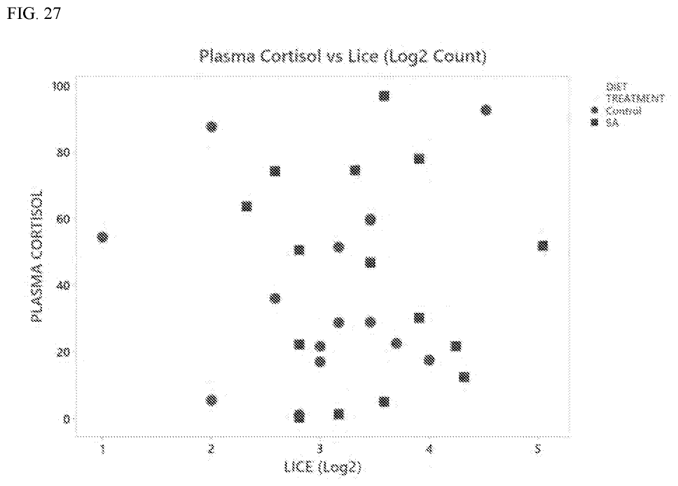

Stress in the case of salmon lice infestation—Cortisol—unsuitable indicator. Plasma cortisol measured is plotted against the intensity of infestation by lice in FIG. 27. There was no correlation between cortisol levels and infestation by lice, neither for all individuals considered together, nor assessed per diet treatment. It is known that lice infestation affects behavior and triggers physiological responses in the salmon. During this experiment, ILAB's staff observed that the fish changed their behavior (intensive jumping) as soon as lice were added to the tanks. The infestation of lice was also significant (2 to 33 per fish; geom. average 10.3). The lack of correlation between the intensity of infestation by lice and cortisol levels indicates either that infestation by lice did not cause significant stress, or, more likely, that cortisol level was an unsuitable indicator of stress in the experimental situation. The explanation for this is probably that cortisol, which is largely “psychologically” controlled, increases sharply in all fish when it is caught, and that the flight/fight response masks finer variations in the same hormone that may have been present before such a harrowing event as to be taken in a net and placed on a measuring board. (Glucose and lactate, which are considered indicators of secondary (heart/gill-related) and tertiary (metabolism-related) stress, were unfortunately not measured after the lice experiment).

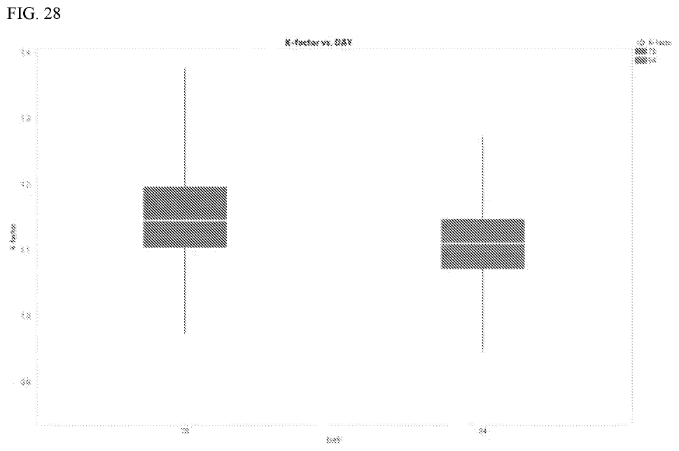

Change in fitness factor during lice infestation Inhibition of growth. and development of poorer fitness factor is an important indicator of physiological stress. Although the growth period in the lice experiment was only 16 days, there was a small but significant decrease in the fitness factor for all the fish taken together (t-test, DF 50.7, t-ratio −2.35, P=0.0113). See FIG. 28.

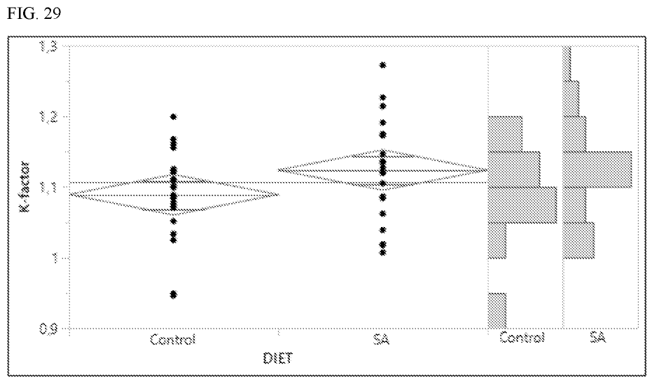

Test of fitness factor against diet (FIG. 29), showed that fish on the SA diet had a significantly higher fitness factor than fish on the control diet after exposure to lice (t-test, DF 42; t ratio 1.54; P=0.0441). The higher fitness factor indicates reduced metabolic stress in fish on the SA diet after infestation of lice. The conclusion about weight benefit from enhanced metabolic efficiency is also supported by the significantly lower lactate levels found in postsmolts on the SA diet

### Histology of Mucous Cells in Case of Lice Infection—Possible Effects of Diet.

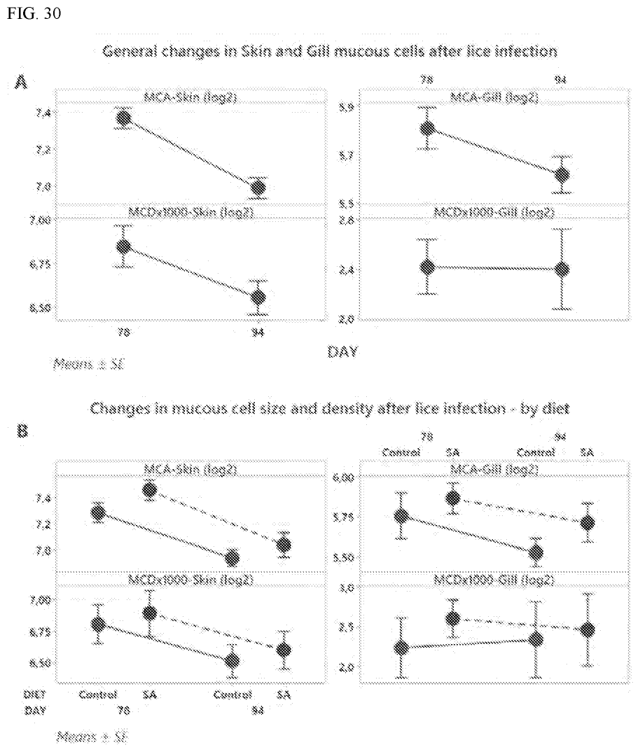

Compared to the growth period before infection, the fitness factor had therefore fallen for all the fish taken together. The results from the periods before infection have shown that fitness and the condition of the epithelium are linked. As expected, we find, for all fish taken together, that both cell size and density on skin epithelium were reduced (MCA; P=0.000; MCD; P=0.06); in gills, cell size was reduced (weakly significant, P=0.099), while the density was not changed (FIG. 30, group A). There were no significant additional effects of diet (FIG. 30, group B).

In this sample, it has thus been shown that the SA diet had an indirect effect on mucus health, through improving the fish's fitness factor, which in turn produces mucus cells with a higher density and average size. The direct additional effect on MCA, found in the preceding growth phase in fresh postsmolt, was qualitatively visible but not statistically significant during the period the lice experiment lasted. Determining whether the additional effect of SA diet on mucus cell size MCA, on top of what comes via better condition, is also significant in case of infection with salmon lice, will require a longer growth period with addition of salmon lice (here: 16 days, from Day 78 to Day 94, against the 28 days of growth for fresh fish, between Day 50 and Day 78; as is known, the experiments were time-limited due to the capacity situation at ILAB).

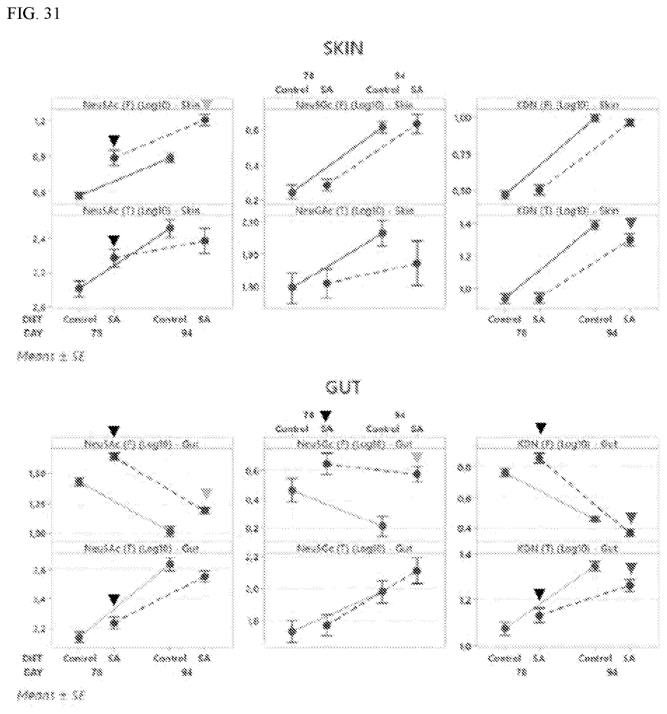

Dynamics of sialic acids in the mucous layer on the skin and in the intestine during infestation by lice. The overview of changes in measured sialic acids before/after the lice experiment is shown in FIG. 31. ANOVA test results comparing the control and SA diet groups' levels of all species of sialic acids from the period before lice infection are shown earlier, while the results after the lice experiment is given in Table 4.

TABLE 4
ANOVA test results, SA diet~Neu5Ac, Neu5Gc and KDN in mucus on skin
and gut, Day 94 (post molt after 16 days of lice infection)
Geom.mean (ÿg/g)

| SA species Mucous Form | | | Control | SA-diet Mean Diff % Diff. | | | DF | F-Value | P-Value |
|---|---|---|---|---|---|---|---|---|---|
| Neu5Ac Skin | | Free | 7.7 | 16.1 | 8.4 | 109% | | | |
| | | Total | 287.1 | 243.3 | NS | NS | 128 | 0.63 | 0.436 |
| | Gut | Free | 10.4 | 15.6 | 5.2 | 50% | 126 | 12.72 | 0.001 |
| | | Total | 4 343.1 | 3 575.2 | NS | NS | 126 | 1.93 | 0.177 |
| Neu5Gc Skin | | Free | 4.1 | 4.3 | NS | NS | 1 | 0.09 | 0.762 |
| | | Total | 111.5 | 80.7 | NS | NS | 1 | 1.35 | 0.254 |
| | Gut | Free | 1.6 | 3.8 | 2.1 | 130% | 116 | 16.53 | 0.001 |
| | | Total | 95.4 | 128.7 | NS | NS | 126 | 1.49 | 0.234 |
| KDN | Skin | Free | 9.9 | 9.2 | NS | NS | 1 | 0.86 | 0.361 |
| | | Total | 24.7 | 20.1 | −4.6 | −19% | 128 | 3.68 | 0.065 |
| | Gut | Free | 2.9 | 2.3 | −0.6 | −19% | 124 | 9.85 | 0.004 |
| | | Total | 22.4 | 18.3 | −4.0 | −18% | 125 | 6.92 | 0.014 |

In general for sialic acids, regardless of diet group, we see the following. On skin: For all three main types of sialic acids, increase of both total and free forms after infestation of lice. In the intestine: For all three main types of sialic acids, a decrease in free and an increase in total forms upon infestation of lice. Specific changes with significant results are as follows.

Neu5Ac: Free form was higher in the SA diet group, both in skin and intestinal mucus, before and after infection. The SA diet group had higher values of total Neu5Ac both on skin and in gut before infection; both differences in total Neu5Ac disappeared after infection.

Neu5Gc: The SA diet group had higher levels of free Neu5Gc in the gut both before and after infection. No other differences in Neu5Gc levels were different either before or after infection.

KDN: Before infection, there were no significant differences in KDN levels in skin between control and SA diets; after infection, the SA group had lower total KDN in skin mucus. Before infection, the SA group had significantly higher levels of both free and total KDN in the intestine; after infection, the SA group had significantly lower levels. SA diet for fish exposed to salmon lice has thus led to an increase in the level of free Neu5Ac on the skin by >100%, and approx. 50% in intestinal mucus. Free Neu5gc in the intestine is increased by 130% during lice infestation. KDN is reduced by 18-19% in skin mucus (total KDN) and intestine (both free and total KDN).

The strong increase in all sialic acid levels in skin mucus is interesting, and that it occurs at the same time as the levels decrease in intestinal mucus; what can be called a “switch” in priorities. The effect of the switch was attenuated (weaker reduction) in the gut for the SA group regarding Neu5Ac and Neu5Gc, but enhanced (greater reduction) for KDN.

In order to investigate what may lie behind the dramatic sialic acid dynamics in FIG. 31, it is therefore not enough to look at which changes are taking place, but which consequences can be linked to them. The disadvantage we have here is that the lice experiment lasted for such a short time, so that only relatively small effects can be studied, such as a marginal decrease in fitness factor. Growth and especially fitness factor can be suitable for this. A response screening of all sialic acid species and forms against fitness factor in the two treatment groups is given in Table 5. This suggests a beneficial effect on fitness from up-regulation especially of Neu5Gc, which tops both lists.

[symbol]

Total Neu5Gc in the intestine had a significant positive correlation with fitness in the control group. At the same time, we see that the SA group succeeds particularly much better in upregulating precisely Neu5Gc in the gut, than the control group did (and the SA group had overall better fitness). In the evaluation of possible interactions between lice infestation and sialic acid responses, one can look at all the individuals together, i.e. without distinguishing between diet groups. This provides greater numerical material to correlate the individual sialic acid species with the confidence factor in the situation of lice infection. As FIG. 21 shows, both the control and SA groups mobilized sialic acid in the skin and in the gut, and had reductions in free forms in the gut—the overall variation was quite similar. This justifies carrying out a grouped response screening (Table 6).

Table 6. Response Screening of Sialic Acids Against Fitness Factor During Lice Infestation, Both Treatment Groups Combined.

[symbol]

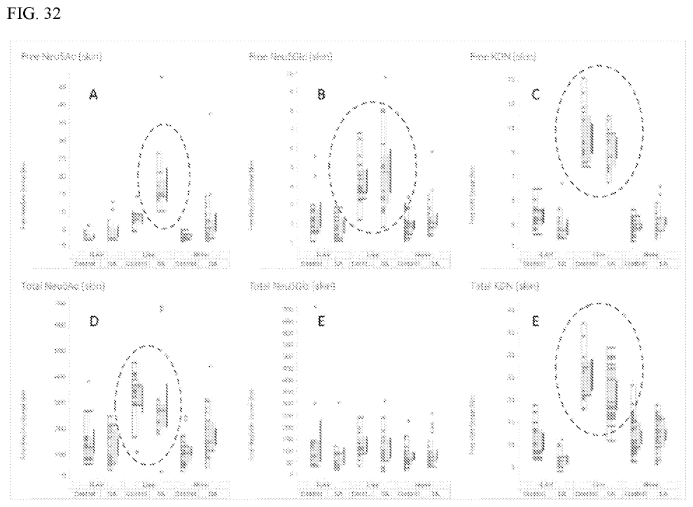

From this overall assessment, it is clear that during the lice experiment, Neu5Gc was associated with higher fitness, as well as free Neu5Ac in the intestine. Among the fiye sialic acid species that score significance, at the top of this list, the SA group stood out with higher levels of both free Neu5Ac and free Neu5Gc in the intestine, number two and three on the list. A final but interesting consideration is that the sialic acid response in the skin during the lice test was unique. Exposure to ILAV produced a far smaller effect, and the situation before infection was also different (FIG. 32).

The increase in free Neu5Ac for the SA group (A in the figure), of free Neu5Gc (B) and total Neu5Ac (D) has been noted previously, but in this arrangement we see that KDN in skin had a particularly strong increase relative to the situations before infection (D78) and during ILAV exposure (D107) (ANOVA, free KDN DF=2.85; F=176.7; P=<0.0001; total KDN DF=2.87, F=88.5, P<0.001). Although both free/total Neu5Ac and free/total Neu5Gc also increased, here we apparently see a particularly clear salmon louse-KDN interaction, which must be to the advantage of one or the other. Relatively speaking, the dramatic physical variation in KDN (C and E in the figure) is little reflected in significance for fitness factor, cf. the response screening above.

Conclusions include the following. In case of salmon lice infection, there is a general up-regulation of sialic acids in the skin (free and bound forms) and of total forms in intestine. Higher levels of Neu5Gc, both in skin and gut, appear to be associated with increased condition in salmon lice infection than the other sialic acids. A significant up-regulation occurred in both diet groups, which indicates that the response is physiologically prioritized, possibly at the expense of other metabolism. SA-supplemented diet significantly strengthened the fish's condition during lice infestation higher levels of free Neu5Ac and free Neu5Gc in intestine.

### VI. Infection by ILA Virus

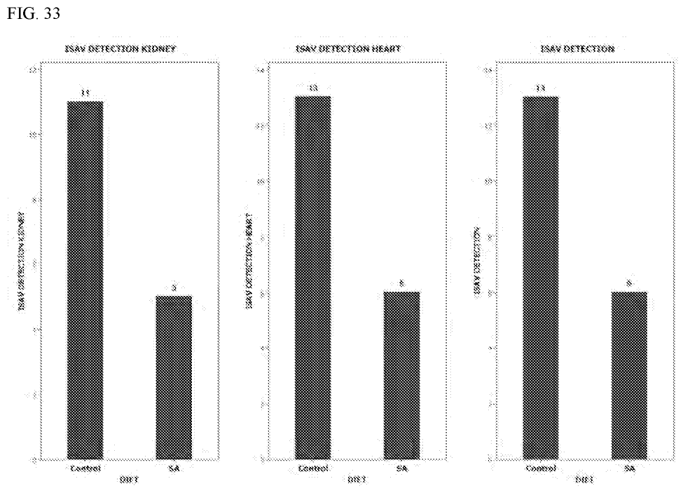

Reduced infection rate of ILA virus on SA diet. At the end of the infection experiment with ILA virus on day 107, infection was examined by PCR analysis in 15 fish from each diet group. A sample of only 2×15 fish is a very small sample to detect differences in, especially in an experimental environment where all fish are guaranteed to be exposed to a significant amount of virus. The results from the infection experiment are shown in FIG. 33. Fish on the SA diet consistently had a lower infection rate both in samples from the kidney, heart and overall, than fish on the control diet.

Data was analyzed by a Chi-square test (Table 7). Despite the very limited numerical basis, a significantly reduced risk of ILAV infection was found for fish on the SA diet among all indicators (detection in heart, kidney and total). This is a simple but strong confirmation of the hypothesis: Free Neu5Ac supplied through diet reduces the risk of infection by ILA virus. The result is therefore highly relevant for all SA-binding viruses that infect salmon and other fish species.

[symbol]

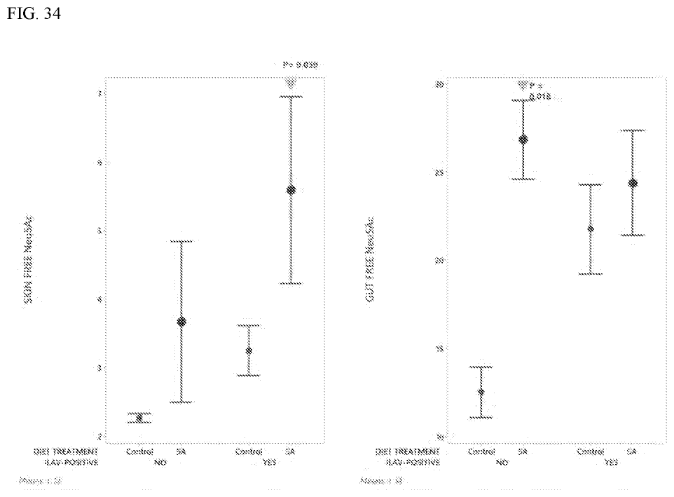

Values of free Neu5Ac (ÿg/g mucus) in the mucus layer on the skin and in the gut from the four groups of infection status and diet from Table 7 are shown in FIG. 34. The low number of fish on the SA diet that tested positive for ILAV (N=6), and ditto on the control diet that tested negative (N=2), weakens the possibility of calculating differences in Neu5Ac levels between the diet groups with statistical significance. Even with these limitations, some differences were clearly significant (indicated in figure). The effect of SA diet on Neu5Ac levels has already been shown in many situations, so perhaps more interesting is the apparent difference in free Neu5Ac in intestinal mucus between healthy and diseased fish in the control group (graph on the right in FIG. 34).

Above all else in the same figure, the control group has significantly lower values of free Neu5Ac than the group on the SA diet. In the intestinal mucus of sick fish from the control group, on the other hand, a mobilization of free Neu5Ac appears to have occurred.

Neu5Ac values in both the skin and intestinal mucus were higher in sick than healthy fish. Fish on the SA diet had higher values than fish on the control diet. The first suggests a response to the viral infection by increased secretion of free Neu5Ac, in both the skin and intestinal epithelium. The second shows that Neu5Ac added via the diet strengthens the fish's ability to exercise this response.

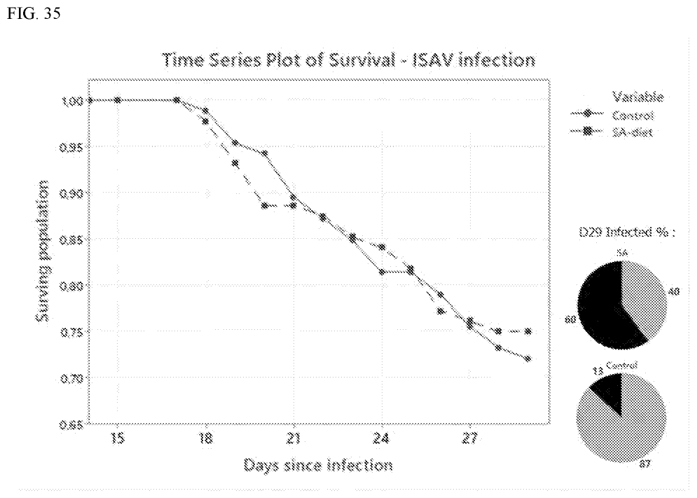

ILAV-infected fish: Survival. The trial could not run as long as desired due to capacity limitations at ILAB, and ended on Day 107—i.e. 29 days after infection. The course of survival during the infection trial with ILAV is shown in FIG. 35. The mortality of fish that had become ill from ILAV during these first 29 days did not differ between the diet groups (binary logistic regression; effect of time P=0.000; additive effect of diet, P=0.414; N dead, survivors 46, 128). A tendency towards lower mortality in the group on the SA diet can possibly be seen towards the end of the experiment, which corresponds to the significantly lower infection frequency among fish on the SA diet at this time (cf. Table 7).

ILAV exposure: Growth and fitness. At the end of the ILAV trial, the total number of surviving fish in the control group was 60, and 66 in the SA group. Fifteen individuals from each group were randomly selected for registration for lab measurements of (sialic acids, histology; the average weights of this selection are reflected in FIG. 19).

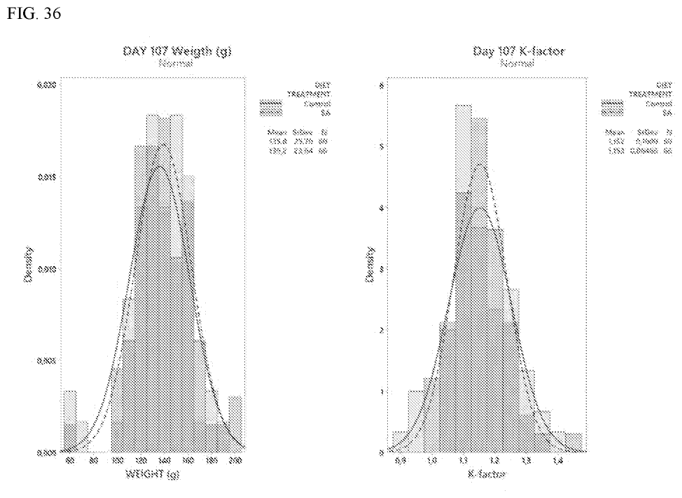

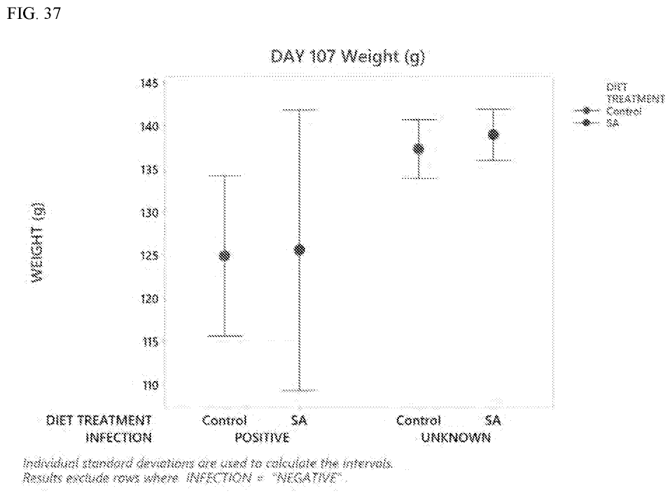

Average weights and average fitness factor for all 60+66 surviving individuals in the two diet groups, with or without ILAV infection, are shown in FIG. 36. Since the weights in ILAB's spreadsheet were given in 1/10th of a gram, and had markings in bold for the 30+30 individuals that had been sent for PCR analysis, these fish were allowed to be grouped from other surviving fish in a restructured data set. Variation in weights for fish with confirmed ILAV and fish with unknown infection status is shown in FIG. 37. Only six fish on the SA diet were infected.

### Summary of Findings

Effects of life history. The life history stage of the fish is of great importance for expected observations of sialic acids in both skin and intestinal mucus. Certain changes have been found to be linked to the fish's physiological changes during smoltification (visible in sampling Day 36, after changing photoperiod and temperature, but before salinity was changed), and some changes later in smoltification can be explained either by physiological changes, or changes in living environment (changes occurred by Day 50, complete smoltification, and environment changed to salt water) (FIG. 4). It has also been shown, as previously known, that life history has a major effect on the epithelium in the skin and gills, especially before/after smoltification.

Effects of size. It has been shown that the fish's condition factor/size has an impact on both the size and the density of mucus cells. This has indirect effects on the histology which must be taken into account in the analysis of the effect of diet and disease on the same responses.

Bioavailability of Neu5Ac added to feed. Free Neu5Ac added to is biologically available through absorption, distribution and metabolism, reflected in increased values of free and total Neu5Ac as well as through interactions with Neu5Gc and KDN. The effect of negative regulation of the fish's own synthesis of Neu5Ac has possibly been observed, and may be significant from a dose-response perspective.

Effect of SA diet on mucus quality. It has been shown that SA diet increases the levels of sialic acids in the fish's mucus layer, and sialic acids are essential building blocks in the most important components of the mucus layers such as mucins, immunoglobulins and other glycosylated molecules that are secreted in the mucus layer. Especially around smoltification and the transition to seawater, the SA diet contributed to rapid and significant improvements in mucus quality (1 month lead in the build-up of free/total Neu5Ac compared to fish on the control diet; higher levels of KDN).

Effect of SA diet on tolerance to salmon lice. SA diet had no effect on attack frequency of copepodites of salmon lice. Fish on the SA diet had reduced stress during heavy lice infestation, reflected in a qualitatively better growth/condition factor than fish on the control diet.

Effect of SA diet on infection by sialic acid-binding virus (model: ILAV). SA diet produced a significantly reduced infection rate of ILA virus (31.5% versus 68.4% by PCR test on day 107). Among all surviving fish at the end of the experiment, the infection rate was 40% in the SA group compared to 87% in the control group. A tendency towards better survival in ILA-infected fish on the SA diet was apparently about to materialize when the experiment was terminated.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method for treating or inhibiting infection by Infectious Salmon Anemia Virus (ISAV), in fish in aquaculture, the method comprising:
administering a composition comprising sialic acid or precursor thereof in an effective concentration to the fish under conditions such that infection by ISAV is treated.

2. A method for prophylaxis of infection by ISAV, in fish in aquaculture, the method comprising:
administering a composition comprising sialic acid or precursor thereof in an effective concentration to the fish under conditions such that infection by ISAV is inhibited.

3. A method for treating or inhibiting infection by Salmon Pancreas Disease Virus (SPDV), in fish in aquaculture, the method comprising:
administering a composition comprising sialic acid or precursor thereof in an effective concentration to the fish under conditions such that infection by SPDV is treated.

4. A method for prophylaxis of infection by SPDV, in fish in aquaculture, the method comprising:
administering a composition comprising sialic acid or precursor thereof in an effective concentration to the fish under conditions such that infection by SPDV is inhibited.

5. A method for treating or inhibiting infection by sea lice, in fish in aquaculture, the method comprising:
administering a composition comprising sialic acid or precursor thereof in an effective concentration to the fish under conditions such that infection by sea lice is treated.

6. A method for prophylaxis of infection by sea lice, in fish in aquaculture, the method comprising:
administering a composition comprising sialic acid or precursor thereof in an effective concentration to the fish under conditions such that infection by sea lice is inhibited.

7. A method for treating or inhibiting infection by Infectious Pancreatic Necrosis Virus (IPNV), in fish in aquaculture, the method comprising:
administering a composition comprising sialic acid or precursor thereof in an effective concentration to the fish under conditions such that infection by IPNV is treated.

8. A method for prophylaxis of infection by IPNV, in fish in aquaculture, the method comprising:
administering a composition comprising sialic acid or precursor thereof in an effective concentration to the fish under conditions such that infection by IPNV is inhibited.

9. A method for treating or inhibiting infection by Piscine Orthoreovirus (PRV), in fish in aquaculture, the method comprising:
administering a composition comprising sialic acid or precursor thereof in an effective concentration to the fish under conditions such that infection by PRV and/or heart and skeletal muscle inflammation is treated.

10. A method for prophylaxis of infection by PRV, in fish in aquaculture, the method comprising:
administering a composition comprising sialic acid or precursor thereof in an effective concentration to the fish under conditions such that infection by PRV and/or heart and skeletal muscle inflammation is inhibited.

11. A method for treating or inhibiting infection by Piscine Myocarditis Virus (PMV), in fish in aquaculture, the method comprising:
administering a composition comprising sialic acid or precursor thereof in an effective concentration to the fish under conditions such that infection by PMV and/or cardiomyopathy syndrome is treated.

12. A method for prophylaxis of infection by PMV, in fish in aquaculture, the method comprising:
administering a composition comprising sialic acid or precursor thereof in an effective concentration to the fish under conditions such that infection by PMV and/or cardiomyopathy syndrome is inhibited.

13. Sialic acid or precursor thereof for use in treating infection by ISAV in a fish in aquaculture.

14. Sialic acid or precursor thereof for use in prophylaxis of ISAV infection in a fish in aquaculture.

15. Sialic acid or precursor thereof for use in treating infection by SPDV in a fish in aquaculture.

16. Sialic acid or precursor thereof for use in prophylaxis of SPDV infection in a fish in aquaculture.

17. Sialic acid or precursor thereof for use in treating infection by sea lice in a fish in aquaculture.

18. Sialic acid or precursor thereof for use in prophylaxis of sea lice infection in a fish in aquaculture.

19. Method or use of any of claims 1 to 18, wherein the sialic acid or precursor thereof is selected from the group consisting of neuraminic acid, n-acetylneuraminic acid (NANA), n-glycolylneuraminic acid (NGNA), N-Acetyl-D-glucosamine (GlcNAC), and N-Acetyl-D-mannosamine.

20. Method or use of claim 19, wherein the sialic acid is NANA.

21. Method or use of claim 19, wherein the sialic acid is NGNA.

22. Method or use of claim 19, wherein the sialic acid is neuraminic acid.

23. Method or use of claim 19, wherein the sialic acid precursor is N-Acetyl-D-mannosamine.

24. Method or use of claim 19, wherein the sialic acid or precursor thereof is a combination of two or more of NANA, NGNA, neuraminic acid and N-Acetyl-D-mannosamine in any combination.

25. Method of any one of claims 1 to 24, wherein the sialic acid or precursor thereof is a purified sialic acid or precursor thereof.

26. Method or use of any one of claims 1 to 25, wherein the sialic acid or precursor thereof is administered orally.

27. Method or use of any one of claims 1 to 26, wherein the sialic acid or precursor thereof is administered topically.

28. Method or use of claim 27, wherein the sialic acid or precursor thereof is administered via a bath.

29. Method or use of any one of claims 1 to 28, wherein the sialic acid or precursor thereof is administered in a salmon feed.

30. Method or use of claim 29, wherein the salmon feed comprises from 0.01% to 2.0% w/w sialic acid or precursor thereof.

31. Method or use of claim 29, wherein the salmon feed comprises from 0.5% to 1.5% w/w sialic acid or precursor thereof.

32. Method of use of any one of claims 29 to 31, wherein the salmon feed further comprises a protein component, a carbohydrate component and a lipid component.

33. Method or use of claim 31, wherein the protein component is fish meal and/or krill meal.

34. Method or use of claim 31, wherein the carbohydrate component is a plant material.

35. Method or use of claim 31, wherein the lipid component is a fish oil.

36. Method or use of any one of claims 1 to 35, wherein the fish is infected with ISAV.

37. Method or use of any one of claims 1 to 35, wherein the fish is infected with SPDV.

38. Method or use of any one of claims 1 to 35, wherein the fish is infected with sea lice.

39. Sialic acid or precursor thereof for use in effecting a health parameter in an aquatic animal, the health parameter selected from the group consisting of:
a) prophylaxis of infection by a sialic acid-binding virus;
b) treatment of infection by a sialic acid-binding virus;
c) amelioration of symptoms caused by sea lice infestation;
d) improvement of skin barrier function by increasing sialic acid content in skin mucous;
e) improvement of gut barrier function by increasing sialic acid content in gut mucous;
f) increasing KDN content in the gut mucous;
g) decreasing stress levels as evidenced by a decrease in lactate levels;
h) treatment or prophylaxis of infection by Aeromonas salmonicida;
i) treatment of symptoms of furunculosis;
j) treatment or prophylaxis of infection by amoebas;
k) stimulation of mucous epithelia cells;
l) improvement in condition factor after smoltifying;
m) improvement of growth after smoltifying; and
n) improvement in length after smoltifying.

40. A method of improving a health parameter in an aquatic animal comprising administering to the aquatic animal sialic acid or a precursor thereof, the health parameter selected from the group consisting of:
a) prophylaxis of infection by a sialic acid-binding pathogen;
b) treatment of infection by a sialic acid-binding pathogen;
c) amelioration of symptoms caused by sea lice infestation;
d) improvement of skin barrier function by increasing sialic acid content in skin mucous;
e) improvement of gut barrier function by increasing sialic acid content in gut mucous;
f) increasing KDN content in the gut mucous;
g) decreasing stress levels as evidenced by a decrease in lactate levels;
h) stimulation of mucous epithelia cells;
i) improvement in condition factor after smoltifying;
j) improvement of growth after smoltifying; and
k) improvement in length after smoltifying.

41. Method or use of any of claims 39 to 40, wherein the sialic acid or precursor thereof is selected from the group consisting of neuraminic acid, n-acetylneuraminic acid (NANA), n-glycolylneuraminic acid (NGNA), N-Acetyl-D-mannosamine, N-acetylglucosamine, mannosamine, fructose and mannose.

42. Method or use of claim 41, wherein the sialic acid is NANA.

43. Method or use of claim 41, wherein the sialic acid is NGNA.

44. Method or use of claim 41, wherein the sialic acid is neuraminic acid.

45. Method or use of claim 41, wherein the sialic acid precursor is N-Acetyl-D-mannosamine.

46. Method or use of claim 41, wherein the sialic acid precursor is N-acetylglucosamine.

47. Method or use of claim 41, wherein the sialic acid precursor is mannosamine.

48. Method or use of claim 41, wherein the sialic acid precursor is mannose.

49. Method or use of claim 41, wherein the sialic acid precursor is fructose.

50. Method or use of claim 41, wherein the sialic acid or precursor thereof is a combination of two or more of neuraminic acid, n-acetylneuraminic acid (NANA), n-glycolylneuraminic acid (NGNA), N-Acetyl-D-mannosamine, N-acetylglucosamine, mannosamine, fructose and mannose in any combination.

51. Method of any one of claims 39 to 50, wherein the sialic acid or precursor thereof is a purified sialic acid or precursor thereof.

52. Method of use of any one of claims 39 to 51, wherein the aquatic animal is a fish.

53. Method or use of claim 52, wherein the fish is a salmon.

54. Method of use of any one of claims 39 to 51, wherein the aquatic animal is a shrimp.

55. Method or use of any one of claims 39 to 54, wherein the sialic acid or precursor thereof is administered orally.

56. Method or use of any one of claims 39 to 55, wherein the sialic acid or precursor thereof is administered in a fish feed.

57. Method or use of claim 56, wherein the fish feed comprises from 0.01% to 2.0% w/w sialic acid or precursor thereof.

58. Method or use of claim 56, wherein the fish feed comprises from 0.05% to 1.5% w/w sialic acid or precursor thereof.

59. Method or use of claim 56, wherein the fish feed comprises from 0.01% to 0.5% w/w sialic acid or precursor thereof.

60. Method or use of claim 56, wherein the fish feed comprises from 0.05% to 0.5% w/w sialic acid or precursor thereof.

61. Method of use of any one of claims 56 to 60, wherein the fish feed further comprises a protein component, a carbohydrate component and a lipid component.

62. Method or use of claim 61, wherein the protein component is fish meal and/or krill meal.

63. Method or use of claim 61, wherein the carbohydrate component is a plant material.

64. Method or use of claim 61, wherein the lipid component is a fish oil.

65. Method or use of any one of claims 39 to 54, wherein the sialic acid or precursor thereof is administered topically.

66. Method or use of claim 65, wherein the sialic acid or precursor thereof is administered via a bath.

67. Method or use of any one of claims 39 to 66, wherein the sialic-acid binding pathogen is selected from the group consisting of a virus, a bacteria and an amoeba.

68. Method or use of claim 67, wherein the sialic-acid binding pathogen is a bacteria.

69. Method or use of claim 68, wherein the bacteria is Aeromonas salmonica.

70. Method or use of claim 69, wherein the administration of the sialic acid or precursor thereof ameliorates one or more symptoms of furunculosis.

71. Method or use of claim 67, wherein the sialic-acid binding pathogen is an amoeba.

72. Method or use of claim 71, wherein the amoeba is a Neoparamoeba.

73. Method or use of claim 72, wherein the administration of the sialic acid or precursor thereof ameliorates one or more symptoms of amoebic gill disease.

74. Method or use of claim 67, wherein the sialic-acid binding pathogen is a virus.

75. Method of use of claim 74, wherein the virus is a non-enveloped icosahedral virus.

76. Method or use of claim 74, wherein the virus is ISAV.

77. Method or use of claim 76, wherein the administration of sialic acid or a precursor thereof ameliorates one or more symptoms associated with ISAV infection.

78. Method or use of claim 74, wherein the virus is SPDV.

79. Method or use of claim 78, wherein the administration of sialic acid or a precursor thereof ameliorates one or more symptoms associated with SPDV infection.

80. Method or use of claim 74, wherein the virus is IPNV.

81. Method or use of claim 76, wherein the administration of sialic acid or a precursor thereof ameliorates one or more symptoms associated with pancreatic disease.

82. Method or use of claim 74, wherein the virus is PRV.

83. Method or use of claim 82, wherein the administration of sialic acid or a precursor thereof ameliorates one or more symptoms associated with heart and skeletal inflammation disease.

84. Method or use of claim 74, wherein the virus is PMV.

85. Method or use of claim 84, wherein the administration of sialic acid or a precursor thereof ameliorates one or more symptoms associated with cardiomyopathy disease.

86. Method or use of claim 74, wherein the virus is Alloherpesviridae.

87. Method or use of any one of claims 74 to 86, where in the aquatic animal is infected with the virus.
